(19) United States
(12) Patent Application Publication
(10) Pub. No.: US 2024/0237520 A1
(43) Pub. Date: Jul. 11, 2024

(54) DOPANTS FOR ELECTRONIC COMPONENTS, THEIR USE IN ELECTRONIC COMPONENTS, AND ELECTRONIC COMPONENTS WITH SUCH DOPANTS

(71) Applicant: Heliatek GmbH, Dresden (DE)

(72) Inventors: Dirk HILDEBRANDT, Ulm (DE); Andre WEISS, Dresden (DE); Gunter MATTERSTEIG, Ulm (DE)

(21) Appl. No.: 18/553,572

(22) PCT Filed: Apr. 4, 2022
(86) PCT No.: PCT/DE2022/100253

§ 371 Date: Oct. 2, 2023

(30) Foreign Application Priority Data
Apr. 6, 2021 (DE) 10 2021 108 497.0

(51) Int. Cl. H10K 85/60; C07D 295/215; H10K 30/50; H10K 30/85; H10K 50/165

(52) U.S. Cl. CPC H10K 85/654; C07D 295/215; H10K 85/615; H10K 30/50; H10K 30/85; H10K 50/165

(57) ABSTRACT

A chemical compound of the general formula I:

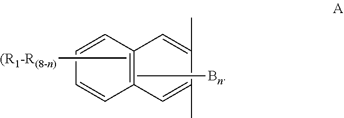

In some aspects, groups B are each independently selected from

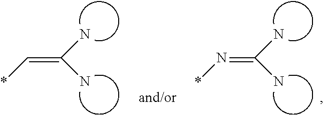 [SMILES: *C=C(N)N ; *N=C(N)N]

where * represents the attachment to group A. In some aspects,

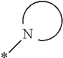 [SMILES: *N]

is a heterocyclic ring having at least one nitrogen atom. In some aspects, R₁-R₍₈₋ₙ₎ are each independently selected from the group consisting of: H, halogen, alkyl, alkenyl, alkynyl, alkoxy, thioalkoxy, aryl, and heteroaryl. In some aspects, R₁-R₍₈₋ₙ₎ form at least one further fused ring on group A. In some aspects, n is a natural number greater than or equal to 1.

### CROSS REFERENCE TO RELATED APPLICATIONS

This application is a U.S. National Phase application under 35 U.S.C. § 371 of International Application No. PCT/DE2022/100253, filed on Apr. 4, 2022, and claims benefit to German Patent Application No. DE 102021108497.0, filed on Apr. 6, 2021, each of which are hereby incorporated by reference in their entirety. The International Application was published in German on Oct. 13, 2022, as WO 2022/214137 A1 under PCT Article 21(2), which is hereby incorporated by reference in its entirety.

### FIELD

The present invention relates to dopants for electronic components, the use of such dopants in electronic components, and electronic components with such dopants.

### BACKGROUND

Photoactive optoelectronic components enable the conversion of electromagnetic radiation into electrical current by making use of the photoelectric effect. A conversion of electromagnetic radiation of this kind requires absorber materials that show good absorption properties. Further optoelectronic components are light-emitting electroluminescent components that emit light when electrical current flows through them. Optoelectronic components comprise at least two electrodes, with one electrode applied to a substrate and the other functioning as counter-electrode. Between the electrodes is at least one photoactive layer, preferably an organic photoactive layer. Further layers, for example transport layers (i.e. charge carrier transport layers), can additionally be arranged between the electrodes.

In organic solar cells, photoactive compounds (absorbers) are typically used in a donor-acceptor system, a heterojunction, where at least the donor and/or the acceptor absorbs electromagnetic radiation. The donor-acceptor system can be designed as a planar heterojunction or as a bulk heterojunction. The absorbers absorb electromagnetic radiation at a specific wavelength, converting photons into excitons that contribute to a photocurrent. The compounds in the donor-acceptor system must have high mobility of charge carriers in order to minimize loss of photocurrent due to recombination of the excitons within the donor-acceptor system. The excitons must be separated into charge carriers at an interface and the charge carriers must leave the photoactive layer before recombination. In order to minimize charge carrier recombination, the conductivity of the layers, especially the transport layers, must be high. The layers used for this purpose, in particular transport layers, electron transport layers or hole transport layers, are doped to increase conductivity.

A structure of an organic solar cell known from the prior art consists of a pin or nip diode (Martin Pfeiffer, “Controlled doping of organic vacuum deposited dye layers: basics and applications”, PhD. thesis TU-Dresden, 1999, and WO2011/161108A1). A pin solar cell consists of a substrate, usually adjoined by a transparent base contact, p layer(s), i layer(s), n layer(s) and a top contact. A nip solar cell consists of a substrate, usually adjoined by a transparent base contact, n layer(s), i layer(s), p layer(s) and a top contact.

The use of doped organic layers or layer systems in organic components, specifically organic solar cells and organic light-emitting diodes, is known. Various materials or material classes have been proposed as dopants, as described in DE102007018456, WO2005086251, WO2006081780, WO2007115540, WO2008058525, WO2009000237 and DE102008051737.

Inorganic dopants such as alkali metals (e.g. cesium) or Lewis acids (e.g. FeCl₃; SbCl₅) are usually disadvantageous in organic matrix materials due to their high diffusion coefficients, as the function and stability of the electronic components is impaired (D. Oeter, Ch. Ziegler Ch, W. Göpel Synthetic Metals (1993) 61 147; Y. Yamamoto et al. (1965) 2015, J. Kido et al. Jpn J. Appl. Phys. 41 (2002) L358). In addition, the reduction potentials of these compounds are often too low to dope technically suitable hole conductor materials. In addition, the extremely aggressive reaction behavior of these dopants makes industrial application more difficult.

Although the dopants disclosed in the prior art are suitable for transport layers in electronic components, there is a need to improve the conductivity of transport layers obtained by doping.

### SUMMARY

In an embodiment, the present disclosure provides a chemical compound of the

general formula I:

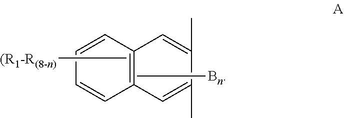

In some embodiments, groups B are each independently selected from

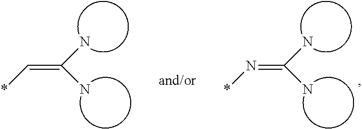 [SMILES: *C=C(N)N ; *N=C(N)N]

where * represents the attachment to group A. In some embodiments,

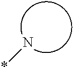 [SMILES: *N]

is a heterocyclic ring having at least one nitrogen atom. In some embodiments, R₁-R₍₈₋ₙ₎ are each independently selected from the group consisting of: H, halogen, alkyl, alkenyl, alkynyl, alkoxy, thioalkoxy, aryl, and heteroaryl. In some embodiments, R₁-R₍₈₋ₙ₎ form at least one further fused ring on group A. In some aspects, n is a natural number greater than or equal to 1.

In another embodiment, the present disclosure provides a use of the chemical compound in the previous embodiment in an electronic component, in particular in an organic electronic component. In some embodiments, the chemical compound is used as a dopant for doping layers in an organic electronic component. In some embodiments, the chemical compound is used in at least one transport layer and/or injection layer.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1: A schematic representation of an exemplary embodiment of the electronic component in cross section;

FIG. 2: A graph showing the electrical conductivity of a transport layer with C60 as matrix material having different proportions of doping with compound 1;

FIG. 3: A graph showing the electrical conductivity of a transport layer with C60 as matrix material having different proportions of doping with compound 5; and

FIG. 4: A graph showing the electrical conductivity of a transport layer with C60 as matrix material having different proportions of doping with 1,8-bis(tetramethylguanidino)naphthalene (TMGN).

### DETAILED DESCRIPTION

The object of the present invention is to provide dopants for doping organic layers of electronic components, which dopants have sufficiently high redox potentials without having any disruptive effects on the matrix material and provide an effective increase in the number of charge carriers in the matrix material.

The object is achieved by the subject matter of the independent claims. Advantageous configurations are apparent from the dependent claims.

The object is especially achieved in that a chemical compound of the general formula I is provided

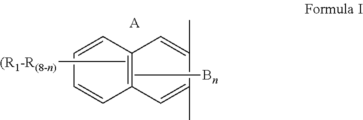

where groups B are each independently selected from

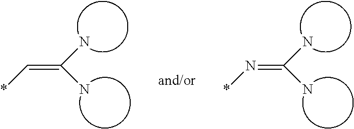 [SMILES: *C=C(N)N ; *N=C(N)N]

where * represents the attachment to group A, wherein

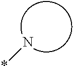 [SMILES: *N]

is a heterocyclic ring having at least one nitrogen atom, R₁-R₍₈₋ₙ₎ are each independently selected from the group consisting of: H, halogen, alkyl, alkenyl, alkynyl, alkoxy, thioalkoxy, aryl, and heteroaryl, and/or R₁-R₍₈₋ₙ₎ form at least one further fused ring on group A, and n is a natural number greater than or equal to 1. The heterocyclic rings

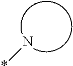 [SMILES: *N]

may comprise further substituents.

In a preferred embodiment of the invention, R₁-R₍₈₋ₙ₎ form at least one further fused ring, particularly preferably one further fused ring, preferably two further fused rings, or preferably three further fused rings.

In a preferred embodiment of the invention, the group A has at least 2, 3, 4, 5 and 6, particularly preferably 2 and/or 3, fused rings.

In a preferred embodiment of the invention, group A is selected from the group consisting of naphthalene, anthracene, phenanthrene, phenalene, tetracene, chrysene, pyrene, pentacene, perylene, benzopyrene, or pentaphene, wherein group A may have further substituents in addition to the n radicals of group B and R₁-R₍₈₋ₙ₎.

In a preferred embodiment of the invention, the heterocyclic rings

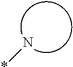 [SMILES: *N]

are preferably formed as four-, five- or six-membered rings.

In a preferred embodiment of the invention

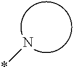 [SMILES: *N]

is a non-aromatic heterocyclic ring having at least one nitrogen atom, preferably having two nitrogen atoms, or preferably having one nitrogen atom and one oxygen atom.

In a preferred embodiment of the invention, R₁-R₍₈₋ₙ₎ are each independently H or alkyl.

The compounds of the invention have advantages compared to the prior art. Advantageously, novel alternative dopants are provided. The dopants are advantageously suitable for doping organic layers, in particular organic transport layers, in electronic components. The compounds advantageously have sufficiently high redox potentials as dopants. Advantageously, the compounds do not have a disruptive effect on the matrix material, in particular on fullerenes. The compounds advantageously contribute to an increase in the number of charge carriers in the matrix material. A particular advantage of the proposed structures is that they are thermally very stable and enable evaporation under high vacuum with a favorable process window between 100° C. and 400° ° C. Another advantage is that the synthesis of the materials can be simple and, above all, also inexpensive; in particular, no explosive and/or hazardous intermediates or starting materials are required. Advantageously, the compounds are stable in air and colorless, and lead to little or no parasitic absorption in solar cells and/or change in the light emitted by light-emitting diodes. The compounds advantageously exhibit outstanding doping properties, preferably when doping transport materials, especially electron transport materials.

A substituent is understood to mean, in particular, the replacement of H by another group. A substituent is understood to mean, in particular, all atoms and atomic groups except hydrogen, preferably a halogen; an alkyl group, where the alkyl group may be linear or branched; an alkenyl group; an alkynyl group; an alkoxy group; a thioalkoxy group; an aryl group; or a heteroaryl group. A halogen is understood to mean, in particular, F, Cl or Br, preferably F.

In a preferred embodiment of the invention, the B groups are arranged on a ring of group A.

In an alternative preferred embodiment of the invention, the B groups are arranged on two rings of group A.

According to a further development of the invention, it is provided that n is at least two, preferably n is 2, 3 or 4, and/or at least two of the B groups are arranged on the same ring of group A.

In an alternative preferred embodiment of the invention, at least two of the B groups are arranged on different, preferably adjacent, rings of the group A.

According to a further development of the invention, it is provided that R₁-R₍₈₋ₙ₎ are each independently H or an alkyl group and/or form at least one further fused ring on group A, and/or the heterocyclic rings

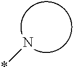 [SMILES: *N]

within a group B are the same.

According to a further development of the invention it is provided that groups B are each independently

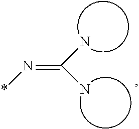 [SMILES: *N=C(N)N]

and/or all groups B are the same. According to a further development of the invention it is provided that groups B are each independently

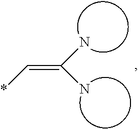 [SMILES: *C=C(N)N]

and/or all groups B are the same.

In a preferred embodiment of the invention, n is at least 2.

In a preferred embodiment of the invention, n is at least 3.

In a preferred embodiment of the invention, n is equal to 2.

In a preferred embodiment of the invention, n is equal to 3.

In a preferred embodiment of the invention, n is equal to 4.

According to a further development of the invention, it is provided that the heterocyclic ring

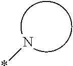 [SMILES: *N]

having at least one nitrogen atom is a four-, five- or six-membered ring, which is substituted or unsubstituted, and/or wherein the heterocyclic ring

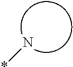 [SMILES: *N]

comprises at least one further heteroatom, preferably at least one further nitrogen atom and/or at least one oxygen atom.

According to a further development of the invention, it is provided that R₁-R₍₈₋ₙ₎ form at least one further ring fused to group A, preferably one further ring fused to group A, preferably two further rings fused to group A, preferably three further rings fused to group A, or preferably four further rings fused to group A, wherein the at least one further fused ring is each independently a homocyclic 5-membered ring or 6-membered ring, which may be substituted or unsubstituted, preferably a benzene ring.

According to a further development of the invention, it is provided that the chemical compound is selected from the group consisting of:

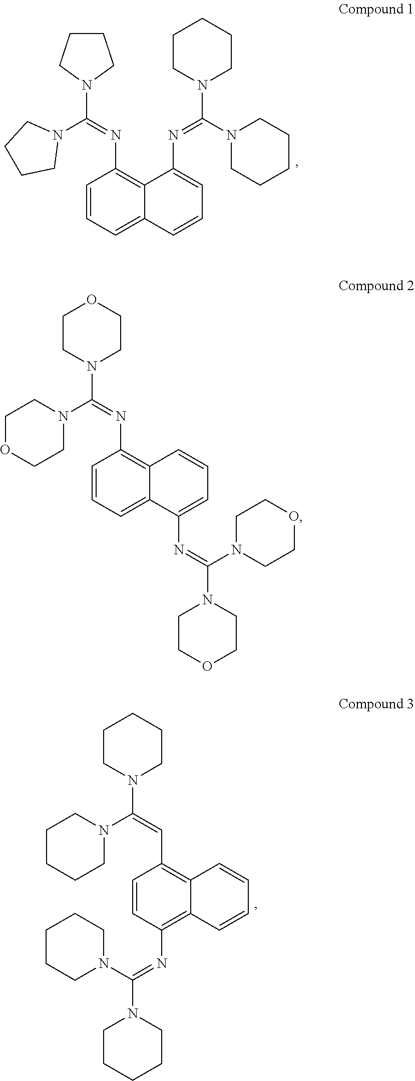 [SMILES: c1cc(N=C(N2CCCCC2)N2CCCCC2)c2c(N=C(N3CCCC3)N3CCCC3)cccc2c1 ; c1cc(N=C(N2CCOCC2)N2CCOCC2)c2cccc(N=C(N3CCOCC3)N3CCOCC3)c2c1 ; C(=C(N1CCCCC1)N1CCCCC1)c1ccc(N=C(N2CCCCC2)N2CCCCC2)c2ccccc12]

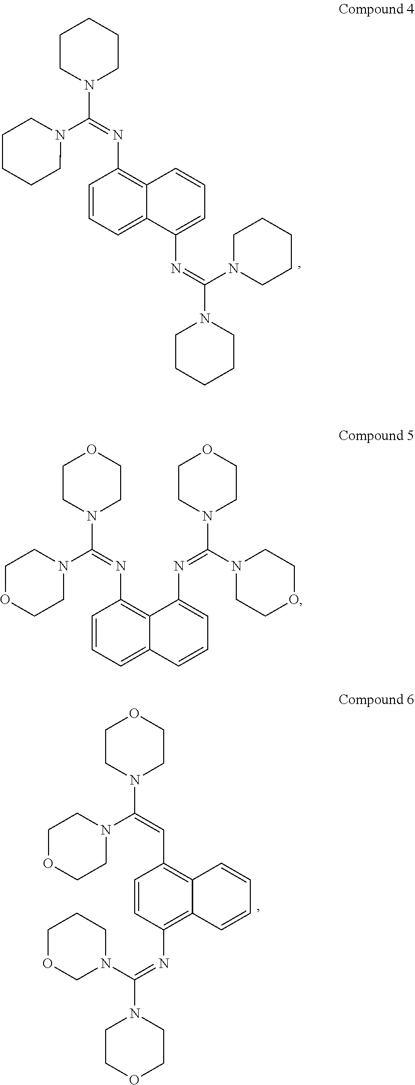 [SMILES: c1cc(N=C(N2CCCCC2)N2CCCCC2)c2cccc(N=C(N3CCCCC3)N3CCCCC3)c2c1 ; c1cc(N=C(N2CCOCC2)N2CCOCC2)c2c(N=C(N3CCOCC3)N3CCOCC3)cccc2c1 ; C(=C(N1CCOCC1)N1CCOCC1)c1ccc(/N=C(/N2CCOCC2)N2CCCOC2)c2ccccc12]

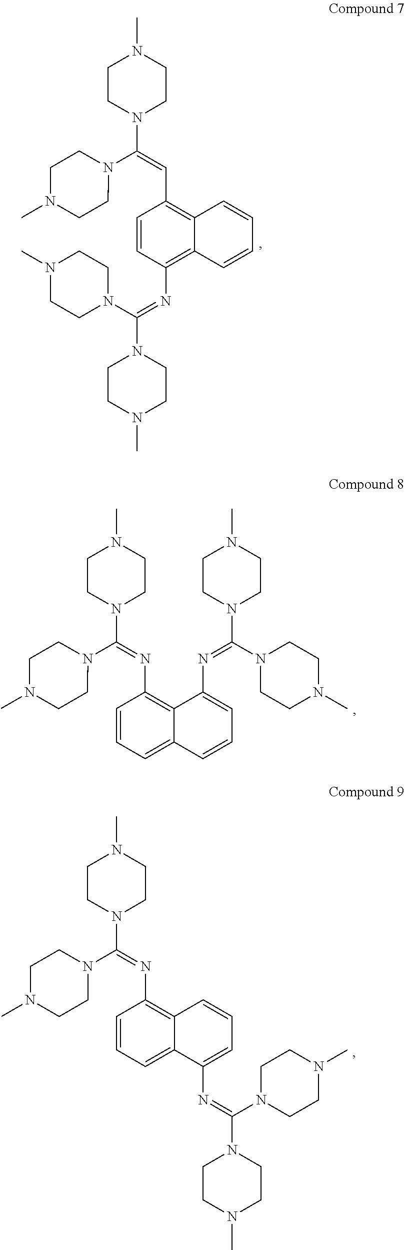

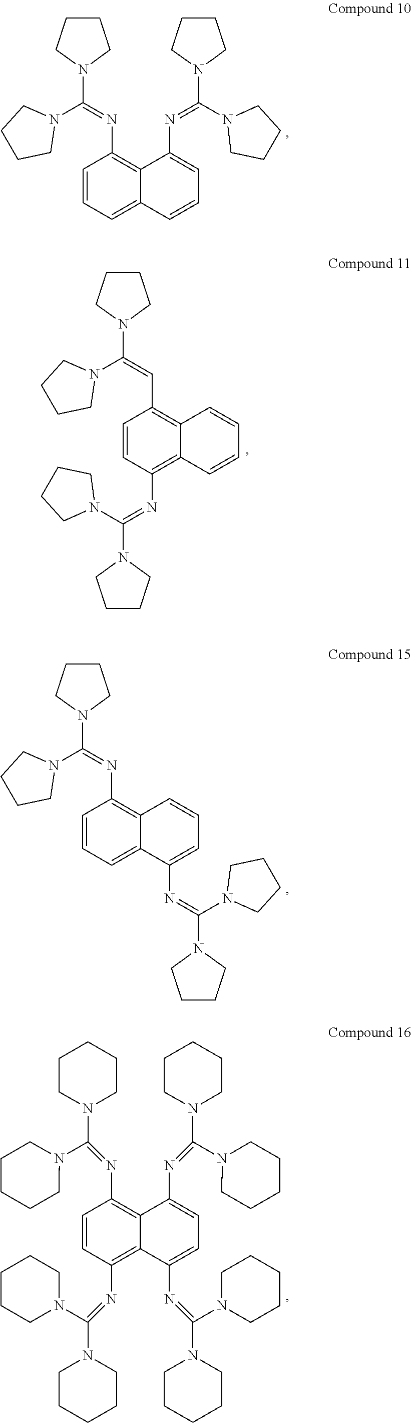

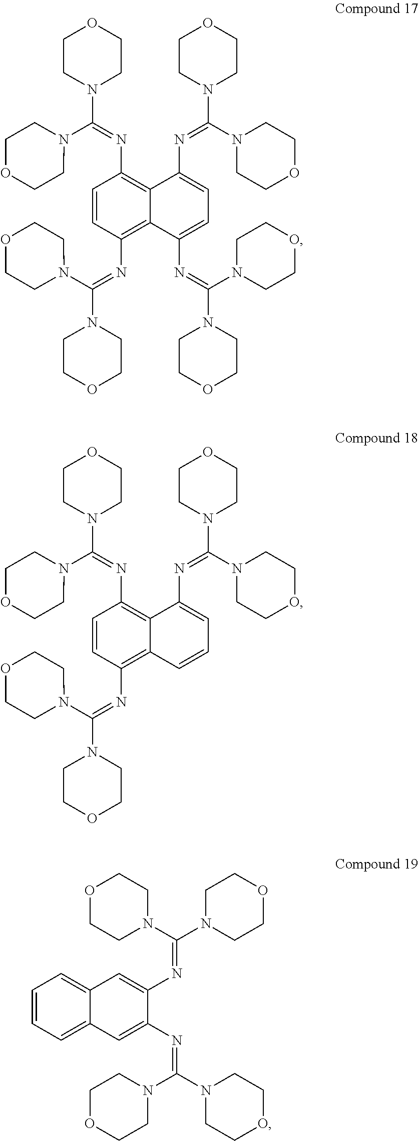 [SMILES: c1cc(N=C(N2CCOCC2)N2CCOCC2)c2c(N=C(N3CCOCC3)N3CCOCC3)ccc(N=C(N3CCOCC3)N3CCOCC3)c2c1N=C(N1CCOCC1)N1CCOCC1 ; c1cc(N=C(N2CCOCC2)N2CCOCC2)c2c(N=C(N3CCOCC3)N3CCOCC3)ccc(N=C(N3CCOCC3)N3CCOCC3)c2c1 ; c1ccc2cc(N=C(N3CCOCC3)N3CCOCC3)c(N=C(N3CCOCC3)N3CCOCC3)cc2c1]

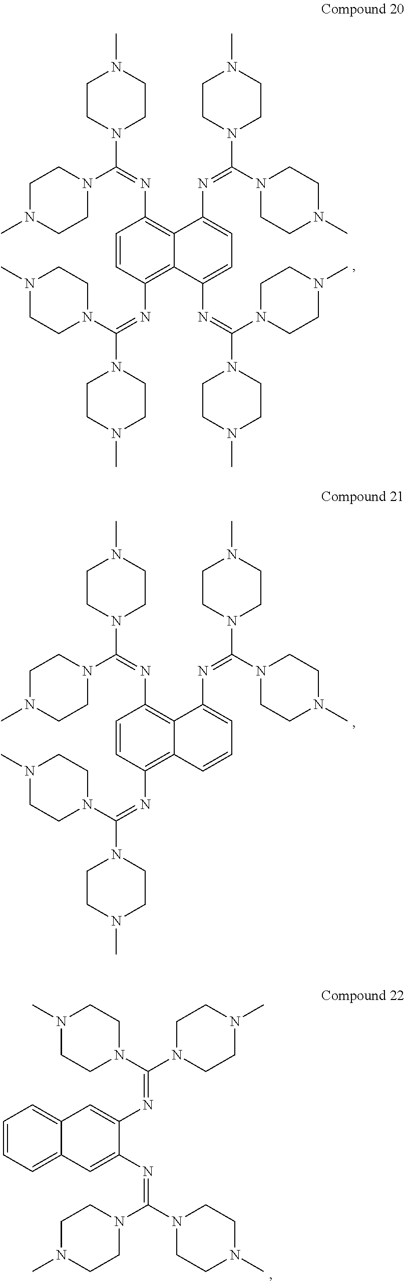

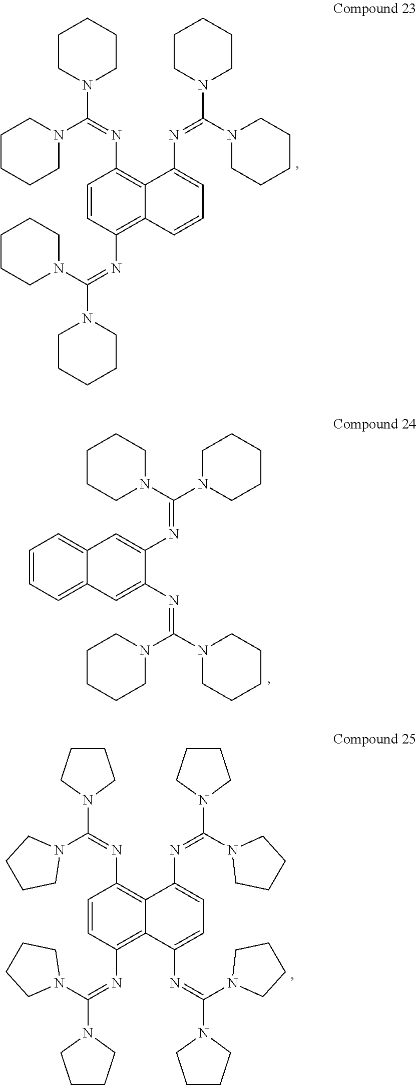 [SMILES: c1cc(N=C(N2CCCCC2)N2CCCCC2)c2c(N=C(N3CCCCC3)N3CCCCC3)ccc(N=C(N3CCCCC3)N3CCCCC3)c2c1 ; c1ccc2cc(N=C(N3CCCCC3)N3CCCCC3)c(N=C(N3CCCCC3)N3CCCCC3)cc2c1 ; c1cc(N=C(N2CCCC2)N2CCCC2)c2c(N=C(N3CCCC3)N3CCCC3)ccc(N=C(N3CCCC3)N3CCCC3)c2c1N=C(N1CCCC1)N1CCCC1]

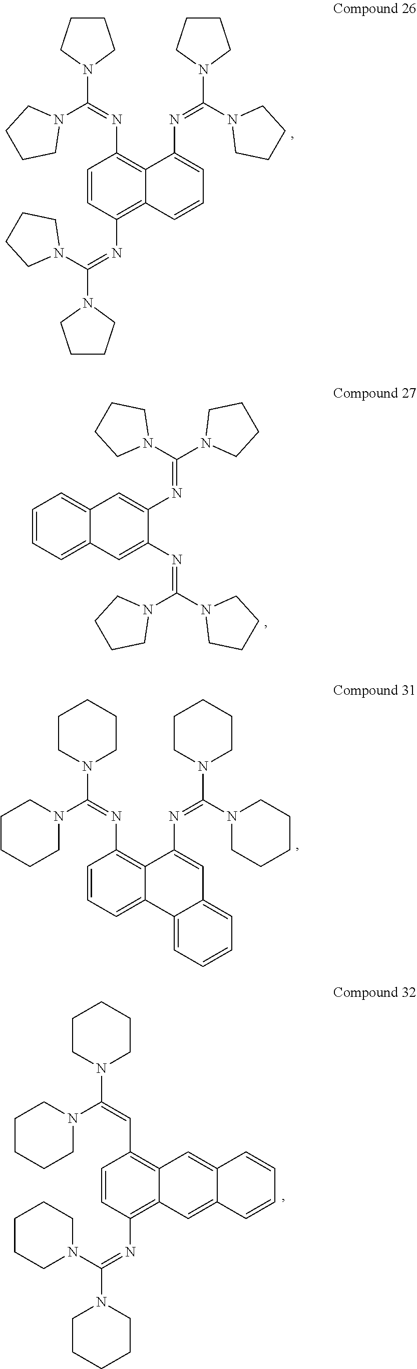 [SMILES: c1cc(N=C(N2CCCC2)N2CCCC2)c2c(N=C(N3CCCC3)N3CCCC3)ccc(N=C(N3CCCC3)N3CCCC3)c2c1 ; c1ccc2cc(N=C(N3CCCC3)N3CCCC3)c(N=C(N3CCCC3)N3CCCC3)cc2c1 ; c1ccc2c(c1)cc(N=C(N1CCCCC1)N1CCCCC1)c1c(N=C(N3CCCCC3)N3CCCCC3)cccc12 ; C(=C(N1CCCCC1)N1CCCCC1)c1ccc(N=C(N2CCCCC2)N2CCCCC2)c2cc3ccccc3cc12]

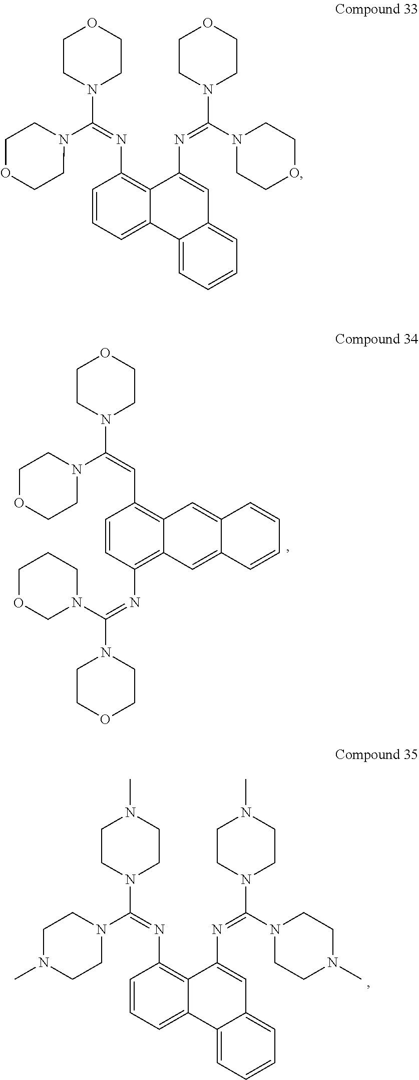

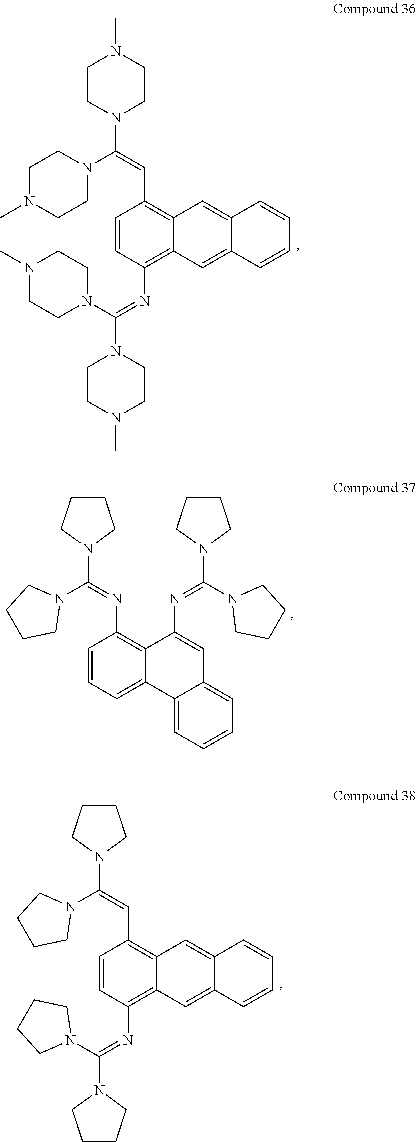 [SMILES: CN1CCN(C(=Cc2ccc(N=C(N3CCN(C)CC3)N3CCN(C)CC3)c3cc4ccccc4cc23)N2CCN(C)CC2)CC1 ; c1ccc2c(c1)cc(N=C(N1CCCC1)N1CCCC1)c1c(N=C(N3CCCC3)N3CCCC3)cccc12 ; C(=C(N1CCCC1)N1CCCC1)c1ccc(N=C(N2CCCC2)N2CCCC2)c2cc3ccccc3cc12]

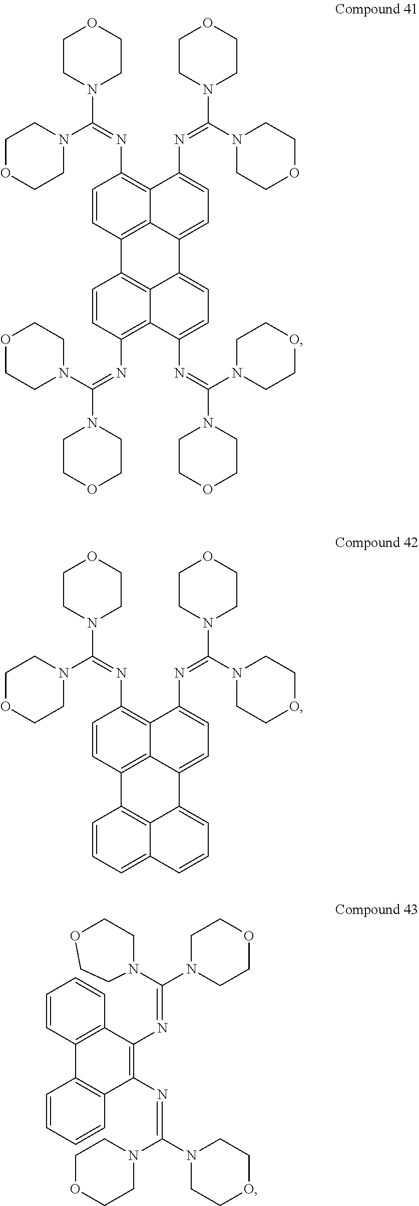 [SMILES: c1cc2c3ccc(N=C(N4CCOCC4)N4CCOCC4)c4c(N=C(N5CCOCC5)N5CCOCC5)ccc(c5ccc(N=C(N6CCOCC6)N6CCOCC6)c(c1N=C(N1CCOCC1)N1CCOCC1)c25)c43 ; c1cc2cccc3c4ccc(N=C(N5CCOCC5)N5CCOCC5)c5c(N=C(N6CCOCC6)N6CCOCC6)ccc(c(c1)c23)c54 ; c1ccc2c(c1)c(N=C(N1CCOCC1)N1CCOCC1)c(N=C(N1CCOCC1)N1CCOCC1)c1ccccc12]

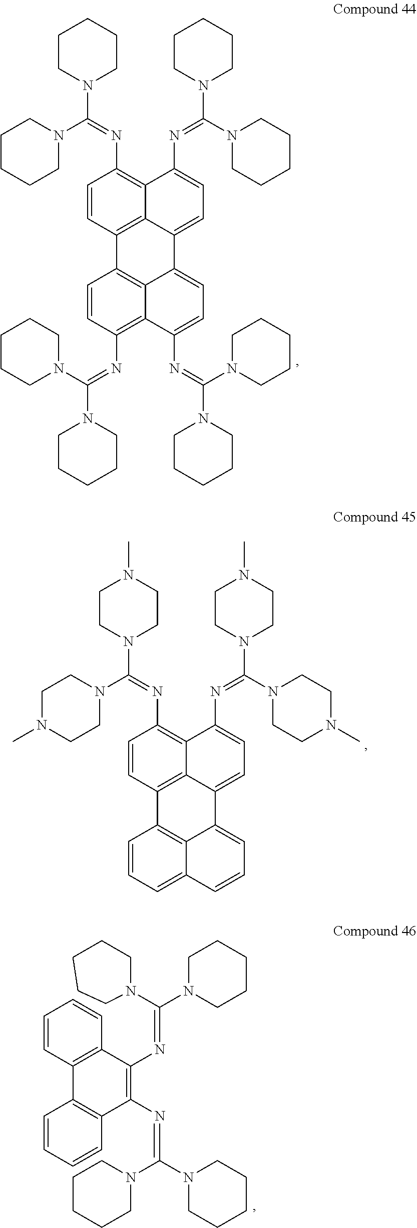

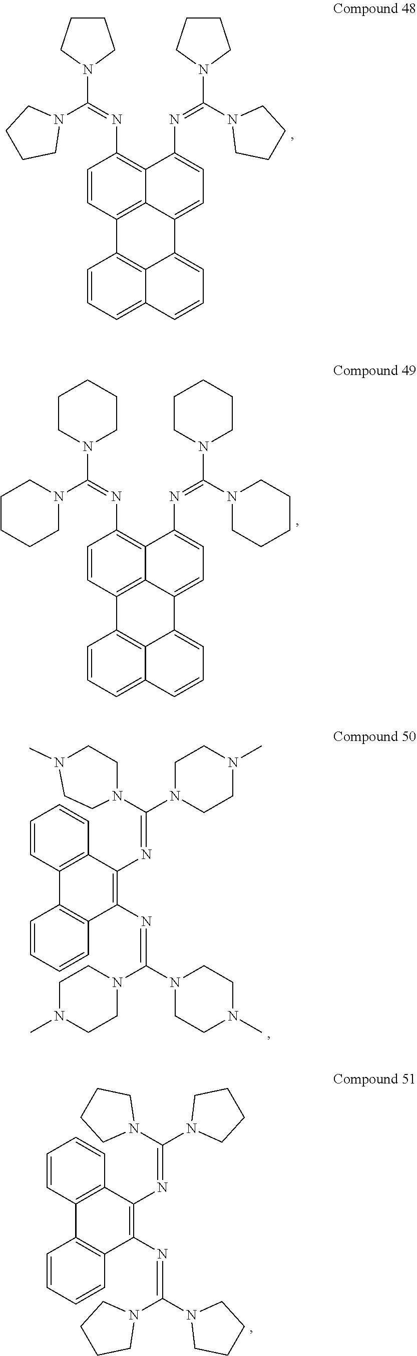

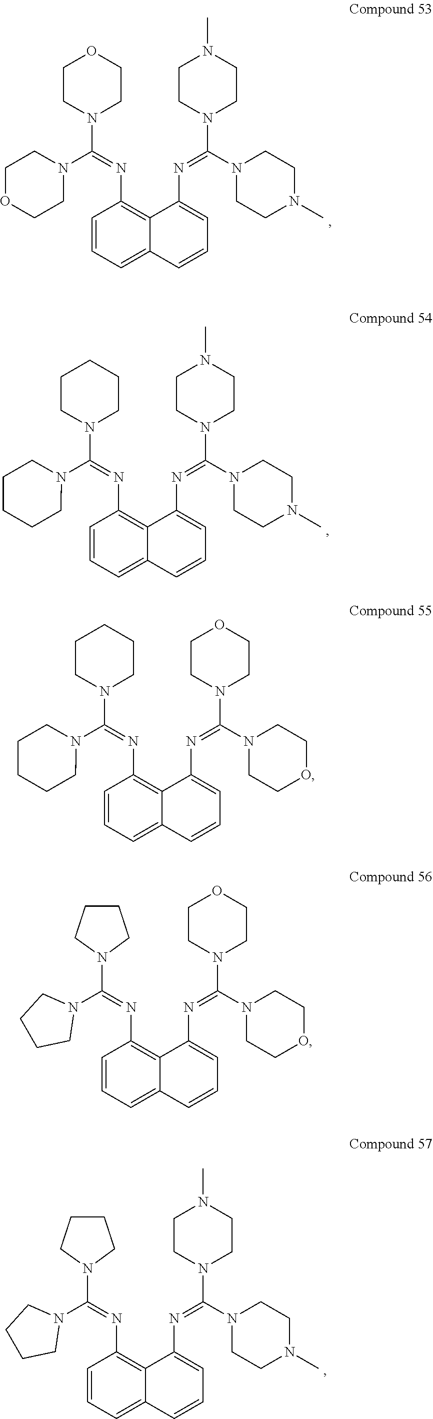 [SMILES: CN1CCN(C(=Nc2cccc3cccc(N=C(N4CCOCC4)N4CCOCC4)c23)N2CCN(C)CC2)CC1 ; CN1CCN(C(=Nc2cccc3cccc(N=C(N4CCCCC4)N4CCCCC4)c23)N2CCN(C)CC2)CC1 ; c1cc(N=C(N2CCCCC2)N2CCCCC2)c2c(N=C(N3CCOCC3)N3CCOCC3)cccc2c1 ; c1cc(N=C(N2CCCC2)N2CCCC2)c2c(N=C(N3CCOCC3)N3CCOCC3)cccc2c1 ; CN1CCN(C(=Nc2cccc3cccc(N=C(N4CCCC4)N4CCCC4)c23)N2CCN(C)CC2)CC1]

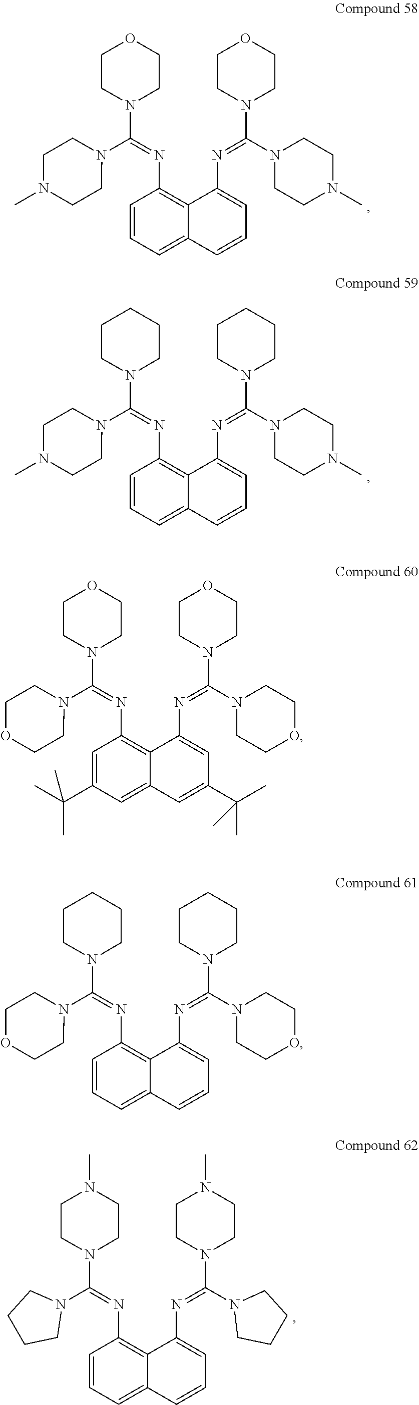

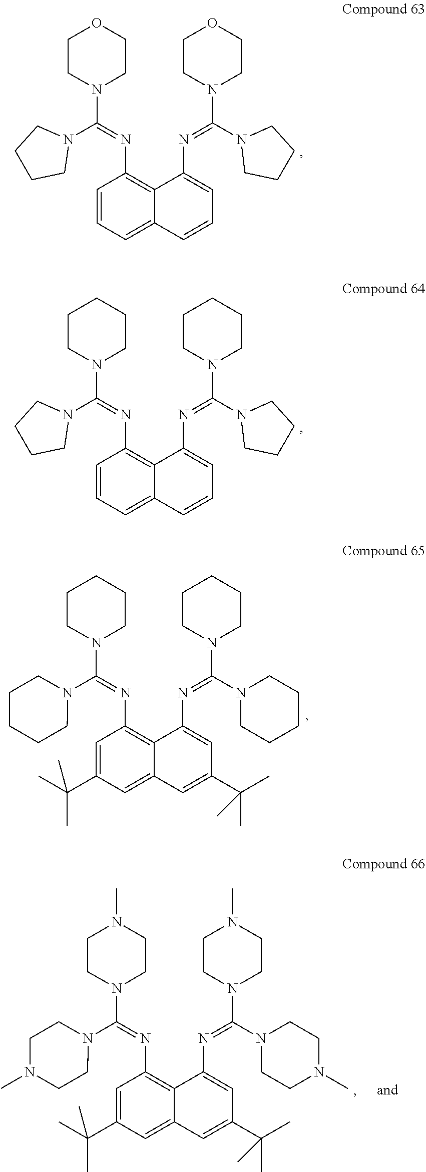

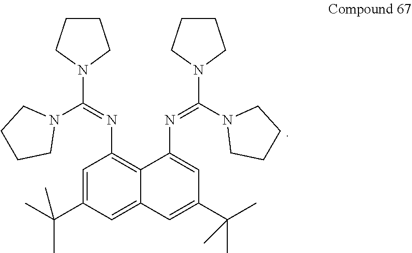 [SMILES: CC(C)(C)c1cc(N=C(N2CCCC2)N2CCCC2)c2c(N=C(N3CCCC3)N3CCCC3)cc(C(C)(C)C)cc2c1]

In a preferred embodiment of the invention, the heterocyclic rings

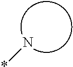 [SMILES: *N]

in a group B are the same.

In a preferred embodiment of the invention, the heterocyclic rings

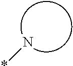 [SMILES: *N]

of all B groups are the same.

In a preferred embodiment of the invention, the chemical compound is selected from the group consisting of:

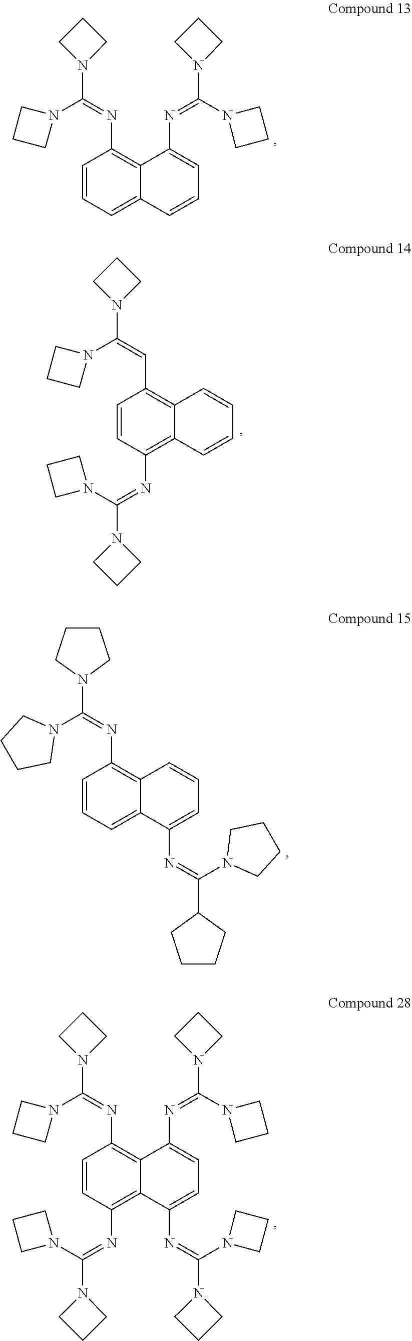

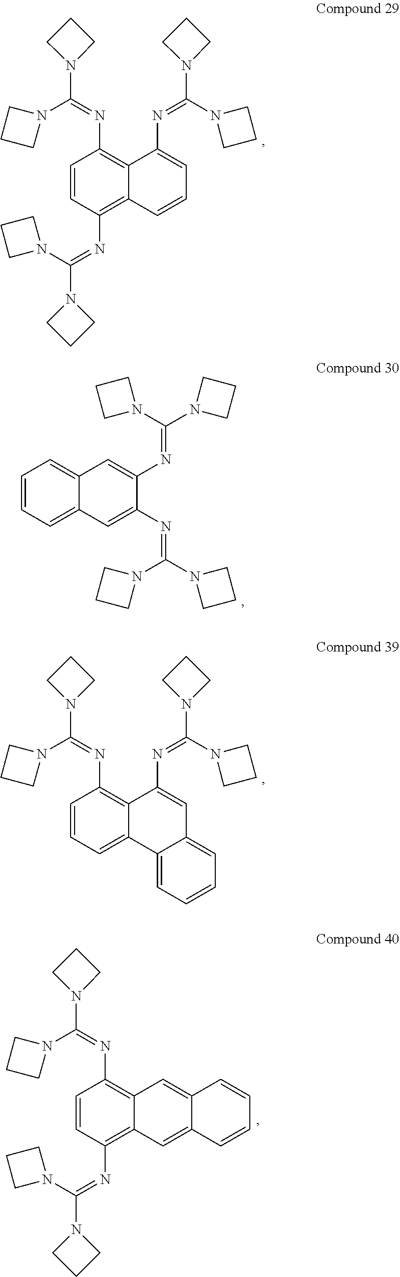 [SMILES: c1cc(N=C(N2CCC2)N2CCC2)c2c(N=C(N3CCC3)N3CCC3)ccc(N=C(N3CCC3)N3CCC3)c2c1 ; c1ccc2cc(N=C(N3CCC3)N3CCC3)c(N=C(N3CCC3)N3CCC3)cc2c1 ; c1ccc2c(c1)cc(N=C(N1CCC1)N1CCC1)c1c(N=C(N3CCC3)N3CCC3)cccc12 ; c1ccc2cc3c(N=C(N4CCC4)N4CCC4)ccc(N=C(N4CCC4)N4CCC4)c3cc2c1]

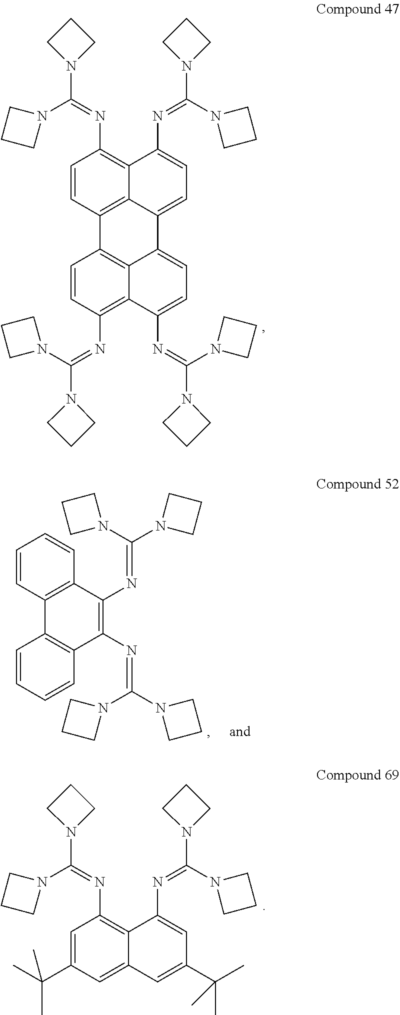 [SMILES: c1cc2c3ccc(N=C(N4CCC4)N4CCC4)c4c(N=C(N5CCC5)N5CCC5)ccc(c5ccc(N=C(N6CCC6)N6CCC6)c(c1N=C(N1CCC1)N1CCC1)c25)c43 ; c1ccc2c(c1)c(N=C(N1CCC1)N1CCC1)c(N=C(N1CCC1)N1CCC1)c1ccccc12 ; CC(C)(C)c1cc(N=C(N2CCC2)N2CCC2)c2c(N=C(N3CCC3)N3CCC3)cc(C(C)(C)C)cc2c1]

In a preferred embodiment of the invention, the chemical compound of the general formula I is mirror- or rotationally symmetrical.

The object of the present invention is also achieved by providing an electronic component, preferably an organic electronic component, having an electrode, a counter-electrode and a layer system between the electrode and the counter-electrode, wherein the layer system comprises at least one organic layer, preferably at least one photoactive layer, and at least one transport layer, in particular according to one of the exemplary embodiments described above. The at least one organic layer and/or the at least one transport layer comprises at least one chemical compound according to the invention. For the electronic component, this gives rise in particular to the advantages already elucidated in connection with the chemical compound according to the invention.

In a preferred embodiment of the invention, the electronic component is an optoelectronic component, preferably an organic optoelectronic component.

In a preferred embodiment of the invention, the at least one transport layer is at least one electron transport layer and/or at least one hole transport layer.

The compounds according to the invention serve in particular as dopants for doping, preferably for n-doping, of an organic matrix material which is preferably used as a charge injection layer, as a hole blocker layer, as an electrode material, as a transport material itself, and/or as a storage material in electronic or optoelectronic components.

In a preferred embodiment of the invention, compounds of the general formula I are used as individual layers, preferably without further admixture.

According to a further development of the invention, the layer system of the electronic component has at least one transport layer, in particular at least one electron transport layer and/or at least one hole transport layer.

In a preferred embodiment of the invention, the transport layer, preferably the electron transport layer and/or the hole transport layer, has a layer thickness of 10 to 100 nm, preferably of 10 to 50 nm, preferably of 10 to 20 nm, or preferably of 20 to 50 nm.

In a preferred embodiment of the invention, the at least one transport layer, preferably the electron transport layer and/or the hole transport layer, is in direct contact with the at least one photoactive layer.

According to a further development of the invention, the proportion of the at least one chemical compound in the at least one organic layer and/or the at least one transport layer is in each case at most 35% by weight, preferably at most 30% by weight, preferably at most 25% by weight, preferably at most 20% by weight, or preferably at most 15% by weight, based on the total weight of the layer.

In a preferred embodiment of the invention, the at least one chemical compound is present in a matrix material of the at least one organic layer and/or the at least one transport layer.

In a preferred embodiment of the invention, the matrix material of the at least one organic layer and/or the at least one transport layer comprises at least one fullerene, preferably C60 or C70, in particular the matrix material consists of at least one fullerene, preferably C60 or C70.

In a preferred embodiment of the invention, the electronic component takes the form of a nip, ni, ip, pnip, pni, pip, nipn, nin, ipn, pnipn, or pipn cell, or of a combination of nip, ni, ip, pnip, pni, pip, nipn, nin, ipn, pnipn, or pipn cells that contain at least one i layer.

The electronic component is preferably designed as a tandem, triple or multiple cell. The photoactive layers of a cell can be designed as a single layer having two or more absorber materials or as a layer system having two or more layers.

An i layer is understood to mean, in particular, an intrinsic undoped layer. One or more i layers may consist here of one material (planar heterojunctions, PHJs) or else of a mixture of two or more materials, called bulk heterojunctions (BHJs) having an interpenetrating network.

According to a further development of the invention, the organic electronic component is an OLED, an organic solar cell, an OFET or an organic photodetector.

In a further embodiment of the invention, the layer system has at least one photoactive layer, preferably an absorber layer, the at least one photoactive layer comprising the at least one compound according to the invention.

In a preferred embodiment of the invention, the organic materials used in the organic layer, in particular the photoactive layer, are small molecules or at least partially polymers.

In a preferred embodiment of the invention, at least one photoactive layer of the organic optoelectronic component comprises small molecules.

Small molecules are understood to mean in particular non-polymeric organic molecules having monodisperse molar masses between 100 and 2000 g/mol that exist in the solid phase at standard pressure (air pressure of the ambient atmosphere) and at room temperature. In particular, the small molecules are photoactive, “photoactive” being understood to mean that the molecules undergo a change of charge state and/or of polarization state when light is supplied. A particular feature of the photoactive molecules is an absorption of electromagnetic radiation within a defined wavelength range, with conversion of absorbed electromagnetic radiation, i.e. photons, to excitons.

In a preferred embodiment of the invention, the acceptor material is a material from the group of fullerenes or fullerene derivatives, preferably C60 or C70, or a PTCDI derivative (perylene-3,4,9,10-bis(dicarboximide) derivative).

In a preferred embodiment of the invention, the electrodes are composed of a metal, preferably Al, Ag, Au or a combination thereof, a conductive oxide, preferably ITO (indium tin oxide), ZnO:Al or another TCO (Transparent Conductive Oxide), a conductive polymer, preferably PEDOT/PSS (poly(3,4-ethylenedioxythiophene)-poly(styrenesulfonate)) or PANI (polyaniline), or formed from a combination of these materials.

In a preferred embodiment of the invention, the photoactive layer has a donor-acceptor system.

In a preferred embodiment of the invention, the at least one donor is an ADA oligomer and/or a BODIPY, and the at least one acceptor is an ADA oligomer and/or a fullerene.

A BODIPY compound is understood to mean in particular a compound of the general formula C₉H₇BN₂F₂, i.e. a compound having a boron difluoride group with a dipyrromethene group, in particular a compound comprising 4,4-difluoro-4-bora-3a,4a-diaza-s-indacene.

In particular, an ADA oligomer is understood to mean a conjugated acceptor-donor-acceptor oligomer (A-D-A′-oligomer) having an acceptor unit (A) and a further acceptor unit (A′), each bonded to a donor unit (D).

In a preferred embodiment of the invention, the at least one transport layer comprises at least one compound of the general formula I as a dopant, in particular as an n-dopant.

The electronic components can be produced in different ways. The layers of the layer system may be applied in liquid form as a solution or dispersion by printing or coating, or by vapor deposition in a vacuum, for example by means of CVD (chemical vapor deposition), PVD (physical vapor deposition) or OVPD (organic vapor phase deposition).

In a preferred embodiment, the compound of the invention and/or a layer comprising the at least one compound of the invention can be deposited by means of vacuum processing, gas-phase deposition or solvent processing, particularly preferably by means of vacuum processing.

In a preferred embodiment of the invention, the transport layer with the at least one compound of the general formula 1 is part of a pn junction connecting a light-absorbing (photoactive) layer to another light-absorbing (photoactive) layer in a tandem solar cell or in a multiple solar cell and/or connecting an electrode to a light-absorbing layer.

The object of the present invention is also achieved by providing for the use of a chemical compound according to the invention in an electronic component, in particular in an organic electronic component, in particular according to one of the exemplary embodiments described above. The use of the chemical compound according to the invention gives rise in particular to the advantages that have already been elucidated in connection with the chemical compound according to the invention and with the electronic component comprising the at least one chemical compound according to the invention.

According to a further development of the invention, the at least one chemical compound according to the invention is used as a dopant for doping layers in an organic electronic component, in particular at least one transport layer and/or injection layer.

In a preferred embodiment of the invention, the dopants for electronic components are dopants for layer systems, preferably for organic layer systems, of an electronic component, preferably an optoelectronic component.

In a preferred embodiment of the invention, at least one transport layer of the layer system comprises at least one compound according to the invention as dopant, preferably as an n-dopant.

In a preferred embodiment of the invention, the at least one chemical compound is used as an n-dopant.

The invention is explained in more detail below with reference to the exemplary embodiments. In particular, it was possible to show that at least one compound according to the invention in a transport layer of a layer system of an electronic component surprisingly results in the conductivity of the layer system being increased.

### WORKING EXAMPLES

### Working Example 1

Working example 1 shows an exemplary embodiment of a synthetic scheme for the synthesis of compounds of the general formula I according to the invention. The synthesis is shown using compound 1 as an example.

### Synthesis of Compound 1:

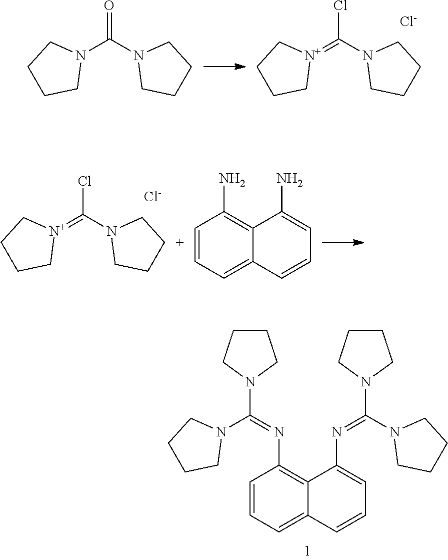

### Synthesis of 1-(chloro(pyrrolidin-1-yl)methylenepyrrolidin-1-ium chloride

13.1 g (78 mmol) of di(pyrrolidin-1-yl)methanone in 150 ml of chloroform are initially charged under an argon atmosphere in a 250 ml three-necked flask equipped with reflux condenser, magnetic stirring bar, argon supply and dropping funnel, and 33.6 ml (384 mmol) of oxalyl dichloride are slowly added dropwise over a period of 1 hour with stirring. The mixture is then heated to 80° C. over 45 minutes with continued stirring and under an argon protective gas atmosphere, and further stirred at this temperature for 20 h. After cooling to room temperature, the solvent is removed on a rotary evaporator and the residue is dried under high vacuum. 14.6 g of a brown solid were obtained, which was used for the next step without further purification.

A suspension of 6.3 g (39 mmol) of 1,8-diaminonaphthalene in a mixture of 160 ml of acetonitrile and 15 ml of triethylamine is initially charged under an argon protective gas atmosphere in a 500 ml three-necked flask equipped with reflux condenser, magnetic stirring bar, argon supply and dropping funnel. While stirring at room temperature, a mixture of 14.6 g (78 mmol) of 1-(chloro(pyrrolidin-1-yl)methylene)pyrrolidin-1-ium chloride, 150 ml of acetonitrile and 65 ml of triethylamine is added via the dropping funnel over a period of 20 minutes. The mixture is stirred at room temperature for 120 h. The precipitate is filtered off and the solvent is removed on a rotary evaporator. The residue (23.1 g of a brown solid) is purified by column chromatography (silica gel; DCM:MeOH:NEt₃ (100:6:0.3; Rf=0.08-0.15)), taken up in dichloromethane, and reprecipitated from n-hexane. 10.77 g (60%) of a pale gray solid were obtained. Compound 1 was characterized as follows:
1H-NMR (400 MHz, CD₂Cl₂) ppm 7.36 (d, 2H, Ar-H), 7.29 (t, 2H, Ar-H), 6.54 (d, 2H, Ar-H), 3.50 (t, 16H, CH₂), 2.00 (t, 16H, CH₂).

In the chemical compound of the general formula I

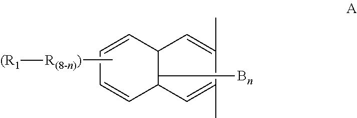

groups B are each independently selected from

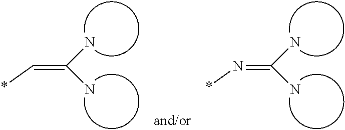 [SMILES: *C=C(N)N ; *N=C(N)N]

where * represents the attachment to group A, wherein

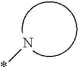 [SMILES: *N]

is a heterocyclic ring having at least one nitrogen atom, R₁-R₍₈₋ₙ₎ are each independently selected from the group consisting of: H, halogen, alkyl, alkenyl, alkynyl, alkoxy, thioalkoxy, aryl, and heteroaryl, and/or R₁-R₍₈₋ₙ₎ form at least one further fused ring on group A, and n is a natural number greater than or equal to 1.

The chemical compounds of the general formula I provide novel dopants for electronic components, in particular for doping photoactive layers and/or transport layers. The compounds advantageously contribute to an increase in the number of charge carriers in the matrix material of photoactive layers and/or of transport layers. Advantageously, the example compounds examined are stable in air and colorless, so that they do not contribute to any parasitic absorption in solar cells and/or to a change in the light emitted by light-emitting diodes.

In one configuration of the invention, n is at least two, preferably n is 2, 3 or 4, and/or at least two of the B groups are arranged on the same ring of group A.

In a further configuration of the invention, R₁-R₍₈₋ₙ₎ are each independently H or an alkyl group and/or form at least one further fused ring on group A, and/or the heterocyclic rings

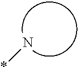 [SMILES: *N]

within a group B are the same.

In a further configuration of the invention, groups B are each independently

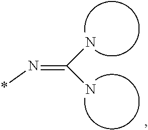 [SMILES: *N=C(N)N]

and/or all groups B are the same.

In a further configuration of the invention, the heterocyclic ring

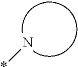 [SMILES: *N]

having at least one nitrogen atom is a four-, five- or six-membered ring, which is substituted or unsubstituted, and/or the heterocyclic ring

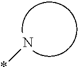 [SMILES: *N]

comprises at least one further heteroatom, preferably at least one further nitrogen atom and/or at least one oxygen atom.

In a further configuration of the invention, R₁-R₍₈₋ₙ₎ form at least one further ring fused to group A, preferably one further ring fused to group A, preferably two further rings fused to group A, preferably three further rings fused to group A, or preferably four further rings fused to group A, wherein the at least one further fused ring is each independently a homocyclic 5-membered ring or 6-membered ring, which may be substituted or unsubstituted, preferably a benzene ring.

The at least one chemical compound is suitable in particular for use in an electronic component 1, in particular in an organic electronic component. In one configuration of the invention, the at least one chemical compound is used as a dopant for doping layers in an organic electronic component, in particular at least one transport layer and/or injection layer, particularly preferably as an n-dopant.

The electronic component 1, preferably the organic electronic component, comprises an electrode 3, a counter-electrode 7 and a layer system 8 between the electrode 3 and the counter-electrode 7, wherein the layer system 8 comprises at least one organic layer 5, preferably at least one photoactive layer, and at least one transport layer 4,6. The at least one organic layer 5 and/or the at least one transport layer 4,6 comprise at least one chemical compound according to the invention.

In a further configuration of the invention, the proportion of the at least one chemical compound in the at least one organic layer 5 and/or the at least one transport layer 4,6 is in each case at most 35% by weight, preferably at most 30% by weight, or preferably at most 25% by weight, based on the total weight of the layer.

### Working Example 2

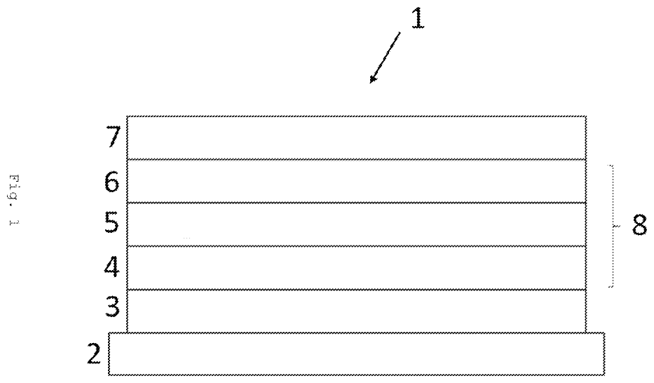

The electronic component 1 comprises a first electrode 3, a second electrode 7, and a layer system 8, the layer system 8 being arranged between the first electrode 3 and the second electrode 7. At least one layer of the layer system 8 comprises at least one compound of the invention. FIG. 1 shows a schematic representation of an exemplary embodiment of the electronic component 1 in cross section. In the present exemplary embodiment, the electronic component 1 is a solar cell, in particular an organic solar cell.

In this exemplary embodiment, the electronic component 1 comprises a substrate 2 composed of glass. A first electrode 3 is arranged on the substrate 2, which is formed, for example, from metal, a conductive oxide, in particular ITO (indium tin oxide), ZnO:Al or another transparent, conductive oxide or polymer such as PEDOT:PSS (poly(3,4-ethylenedioxythiophene)-poly(styrenesulfonate)) or PANI (polyaniline). A layer system 8 is arranged on the first electrode 3 with a transport layer 4, formed as an n-doped electron-transporting layer 4 (ETL). The photoactive layer 5 is arranged on the transport layer 4, which comprises at least one donor and one acceptor material, in particular with a p-conducting donor material and an n-conducting acceptor material, for example C60 fullerene, which together form a donor-acceptor system, either as a flat heterojunction or as a bulk heterojunction. The photoactive layer 5 may also consist of more than one layer, in particular of a donor and acceptor layer, so that a planar, photoactive donor-acceptor junction is formed. Disposed atop that are a transport layer 6, in particular a p-doped hole transport layer 6 (HTL), and the electrode 7 composed of aluminum. The transport layer 6 may also be in the form of an electron conduction transport layer. In this exemplary embodiment, the electron-transporting layer 4 (ETL) is n-doped with the compound 1. In a further configuration, the transport layer 4 can be designed as a hole conduction transport layer.

The electronic component 1 used to measure the conductivity is constructed as follows:

| Material | Thickness/nm | Mixing ratio | |
|---|---|---|---|
| Glass | 10⁶ | — | |
| ITO | 150 | — | |
| C60:compound 1 | 10 | 4:1 | (weight) |
| C60 | 10 | — | |
| [Absorber]:C60 | 30 | 42:53 | (Volume) |
| [NHT49] | 5 | — | |
| [NHT49]:NDP9 | 30 | 9:1 | (weight) |

| | | |
|---|---|---|
| Al | 100 | — |

ITO serves as an electrode and the adjacent fullerene C60 as an electron transport layer (ETL), followed by the photoactive layer C60 as electron acceptor material and the respective compound according to the invention as hole acceptor material (donor material). NDP9 is a commercial p-dopant from Novaled GmbH. NHT49 is a commercial hole conductor from Novaled GmbH.

### Working Example 3

Doping of electronic components 1 according to working example 2 with compound 1, compound 5, and with the comparative material TMGN (1,8-bis(tetramethylguanidino)maphthalene).

In this working example, the transport layer 4,6 is an electron transport layer with n-doping by means of compound 1, compound 5 or the comparative material TMGN. The compounds were investigated with respect to their effect as n-dopants. The compounds were applied using the C60 electron transport material as matrix material and the conductivity of the doped layers was investigated.

Evaporation test compound 1 and comparative material TMGN

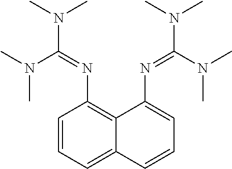

### 1,8-Bis(tetramethylguanidino)naphthalene (TMGN)

Compound 1 and compound 5, each according to the invention, and the comparative material TMGN were each evaporated under high vacuum conditions. The temperature at which a constant deposition rate T of 0.2 A/s is produced on a substrate was determined.

| | Compound 1 | TMGN |
|---|---|---|
| T [0.2 A/s] | >120° C. | 65° C. |

It can be seen that the comparative material TMGN already evaporates at 65° C. At this temperature, evaporation in a vacuum makes it difficult to maintain a constant deposition rate on an uncooled substrate. In addition, significant amounts are likely to be deposited not on the substrate but at undesirable locations within the vacuum system. Compound 1 according to the invention, on the other hand, only evaporates above 120° C., whereby a uniform deposition rate on the substrate can be set which deposition rate can be controlled without increased technical effort.

To dope the transport layers, the electron transport material C60 was co-evaporated with compound 1, compound 5 or the comparative material TMGN and the conductivity of the doped layer was investigated. The transport layer comprises compound 1, compound 5 or the comparative material TMGN as matrix material in each case in proportion to the main proportion of the material C60.

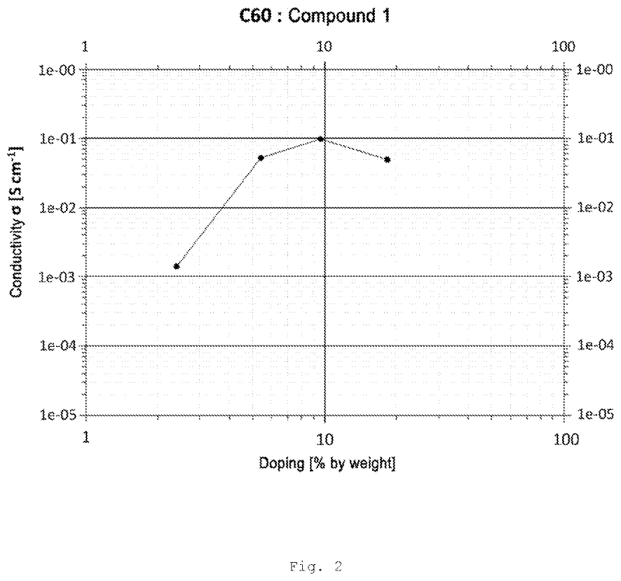

In an exemplary embodiment, FIG. 2 shows the electrical conductivity of a transport layer with C60 as matrix material having different proportions of doping with compound 1. In this exemplary embodiment, the transport layer is an electron transport layer. The conductivity of C60 layers each having 2.5% by weight, 5% by weight, 10% by weight and 20% by weight doping with compound 1 was measured.

The conductivity of the transport layer increases as a function of the proportion of doping with compound 1 according to the invention and reaches a value of 1×10⁻¹ S·cm⁻¹ at a proportion of 10% by weight. In contrast, the conductivity of a layer consisting only of C60 is 1×10⁻¹⁰ S·cm⁻¹ to 1×10⁻⁸ S·cm⁻¹.

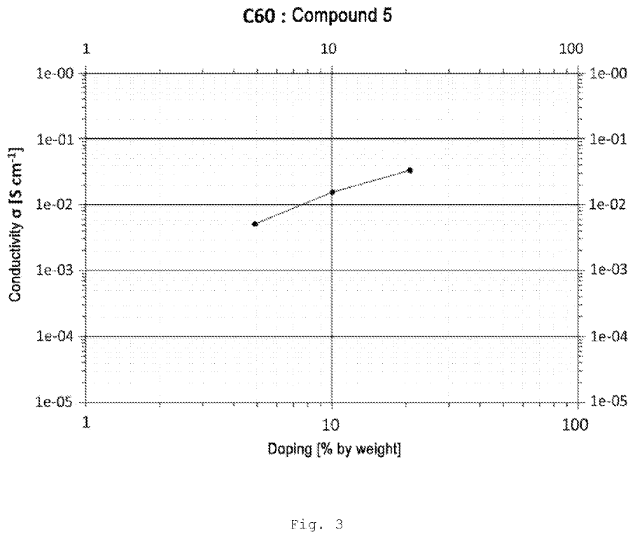

In an exemplary embodiment, FIG. 3 shows the electrical conductivity of a transport layer with C60 as matrix material having different proportions of doping with compound 5. The conductivity of C60 layers each having 5% by weight, 10% by weight and 20% by weight doping with compound 5 was measured.

The conductivity of the transport layer increases as a function of the proportion of doping with compound 5 according to the invention. When 10% by weight of compound 5 is added, the electrical conductivity is 1.7×10⁻² S·cm⁻¹ and, at an addition of 20% by weight, 3×10⁻² S·cm⁻¹.

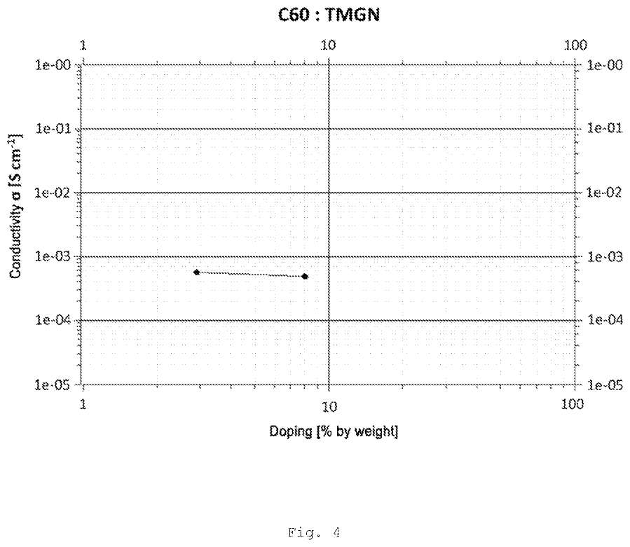

In an exemplary embodiment, FIG. 4 shows the electrical conductivity of a transport layer having C60 as matrix material having different proportions of doping with the comparative material TMGN. The conductivity of C₆₀ layers having 3% by weight and 8% by weight doping with TMGN was measured.

The conductivity of the transport layer with a doping of 8% by weight with the comparative material TMGN is 4.8×10⁻⁴ S·cm⁻¹.

When using compound 1 compared to the comparative material TMGN, conductivity of the C60 layer that is higher by a factor of 200 can be obtained (FIGS. 2 and 4). When using compound 5 compared to the comparative material TMGN, conductivity of the C60 layer that is higher by a factor of 35 can be obtained (FIGS. 3 and 4).

The chemical compounds of the general formula I increase the conductivity of a layer of a layer system of an electronic component. In particular, it is shown that doping a matrix material of an organic layer and/or a transport layer, in particular a C60-containing matrix material, with a compound according to the invention, significantly increases the conductivity of these layers.

### STATEMENTS REGARDING INCORPORATION BY REFERENCE AND VARIATIONS

All references throughout this application, for example, patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A chemical compound of the general formula I 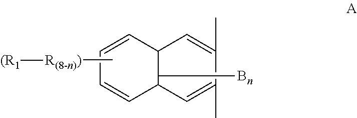
where groups B are each independently selected from 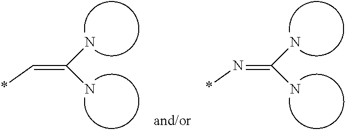 [SMILES: *C=C(N)N ; *N=C(N)N]
where * represents the attachment to group A, wherein 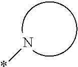 [SMILES: *N]
is a heterocyclic ring having at least one nitrogen atom, R₁-R₍₈₋ₙ₎ are each independently selected from the group consisting of: H, halogen, alkyl, alkenyl, alkynyl, alkoxy, thioalkoxy, aryl, and heteroaryl, and/or R₁-R₍₈₋ₙ₎ form at least one further fused ring on group A, and n is a natural number greater than or equal to 1.

2. The chemical compound as claimed in claim 1, wherein n is at least two, preferably n is 2, 3 or 4, and/or wherein at least two of the B groups are arranged on the same ring of group A.

3. The chemical compound as claimed in claim 1, wherein R₁-R₍₈₋ₙ₎ are each independently H or an alkyl group and/or form at least one further fused ring on group A, and/or the heterocyclic rings 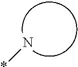 [SMILES: *N]
within a group B are the same.

4. The chemical compound as claimed in claim 1, wherein group B is each independently 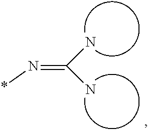 [SMILES: *N=C(N)N]
and/or all groups B are the same.

5. The chemical compound as claimed in claim 1, wherein the heterocyclic ring 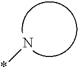 [SMILES: *N]
having at least one nitrogen atom is a four-, five- or six-membered ring, which is substituted or unsubstituted, and/or wherein the heterocyclic ring 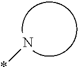 [SMILES: *N]
comprises at least one further heteroatom, preferably at least one further nitrogen atom and/or at least one oxygen atom.

6. The chemical compound as claimed in claim 1, wherein R₁-R₍₈₋ₙ₎ form at least one further ring fused to group A, preferably one further ring fused to group A, preferably two further rings fused to group A, preferably three further rings fused to group A, or preferably four further rings fused to group A, wherein the at least one further fused ring is each independently a homocyclic 5-membered ring or 6-membered ring, which may be substituted or unsubstituted, preferably a benzene ring.

7. The chemical compound as claimed in claim 1, wherein the chemical compound is selected from the group consisting of:

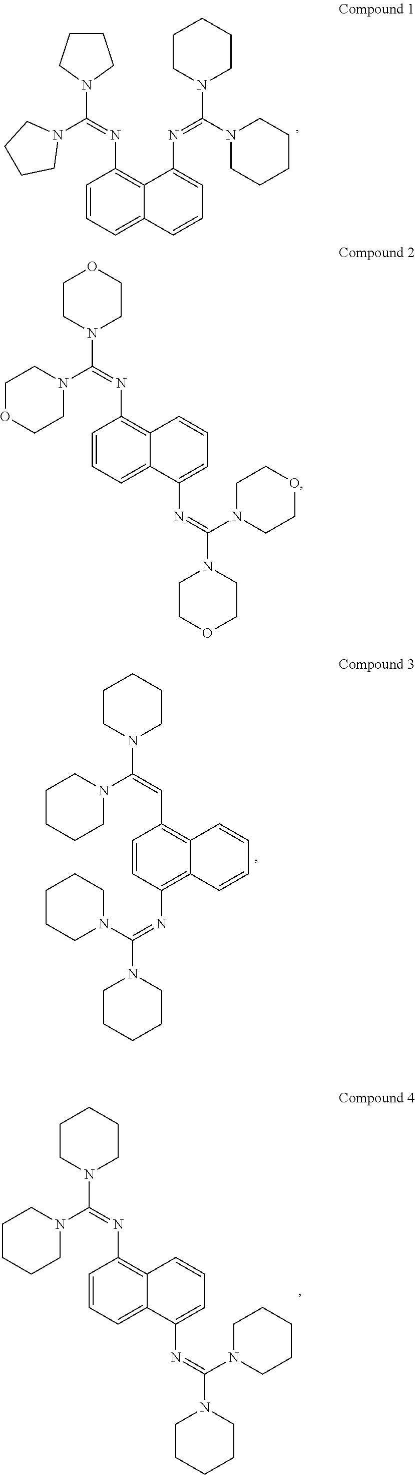

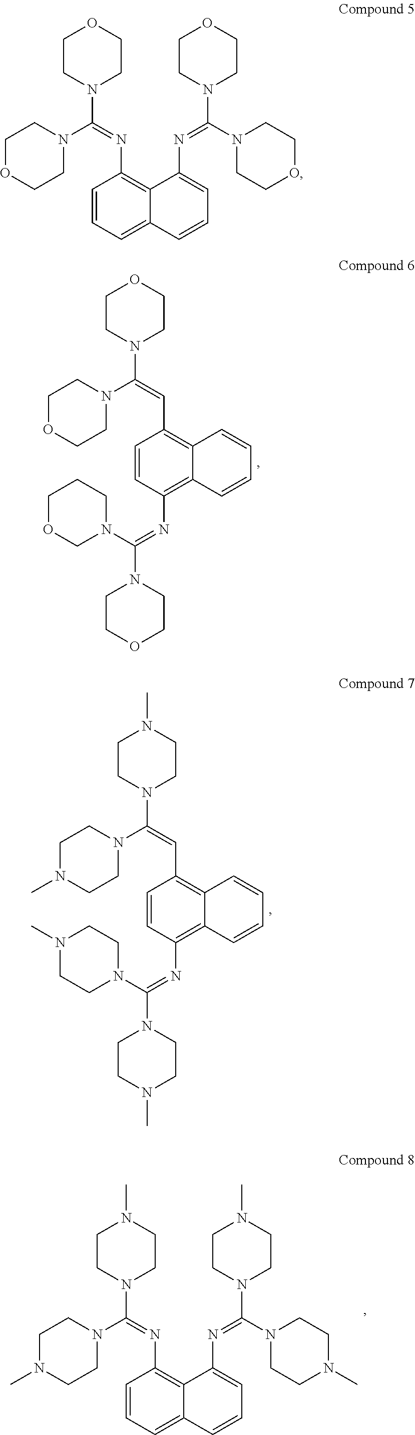

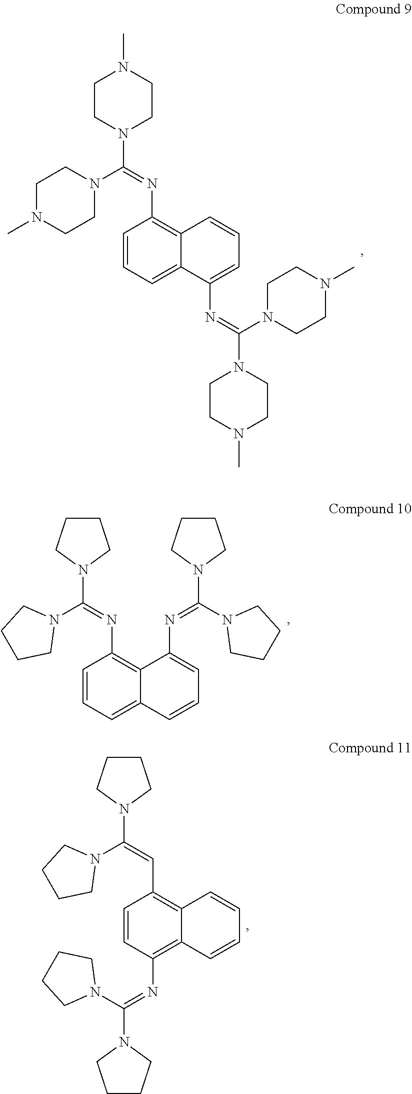

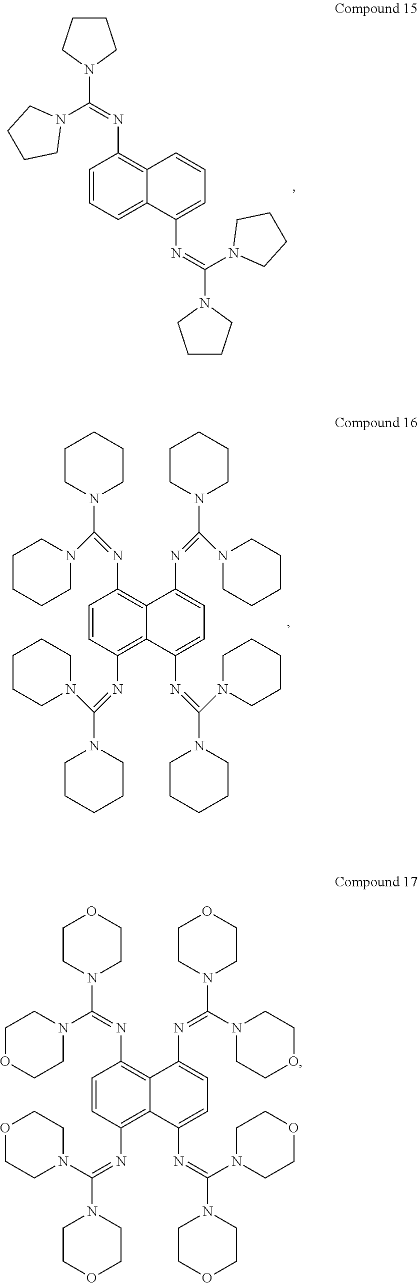 [SMILES: c1cc(N=C(N2CCCC2)N2CCCC2)c2cccc(N=C(N3CCCC3)N3CCCC3)c2c1 ; c1cc(N=C(N2CCCCC2)N2CCCCC2)c2c(N=C(N3CCCCC3)N3CCCCC3)ccc(N=C(N3CCCCC3)N3CCCCC3)c2c1N=C(N1CCCCC1)N1CCCCC1 ; c1cc(N=C(N2CCOCC2)N2CCOCC2)c2c(N=C(N3CCOCC3)N3CCOCC3)ccc(N=C(N3CCOCC3)N3CCOCC3)c2c1N=C(N1CCOCC1)N1CCOCC1]

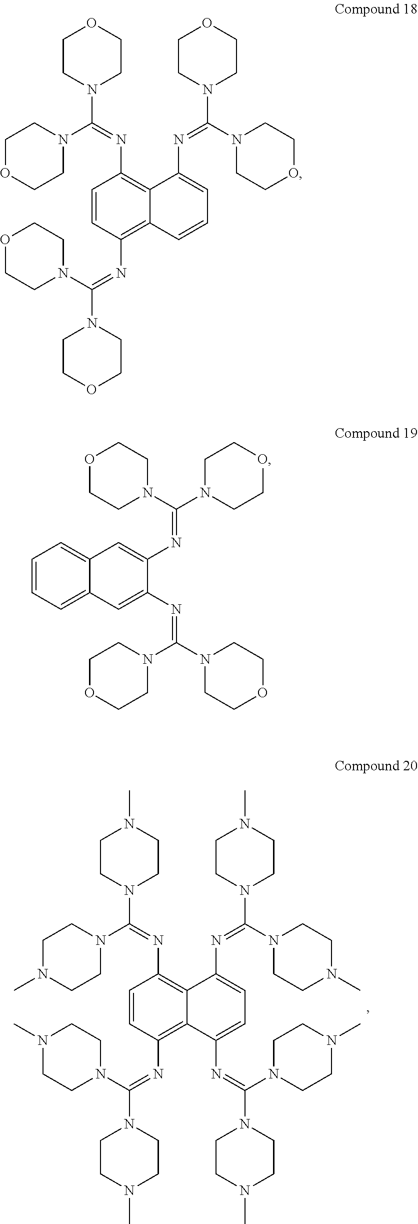

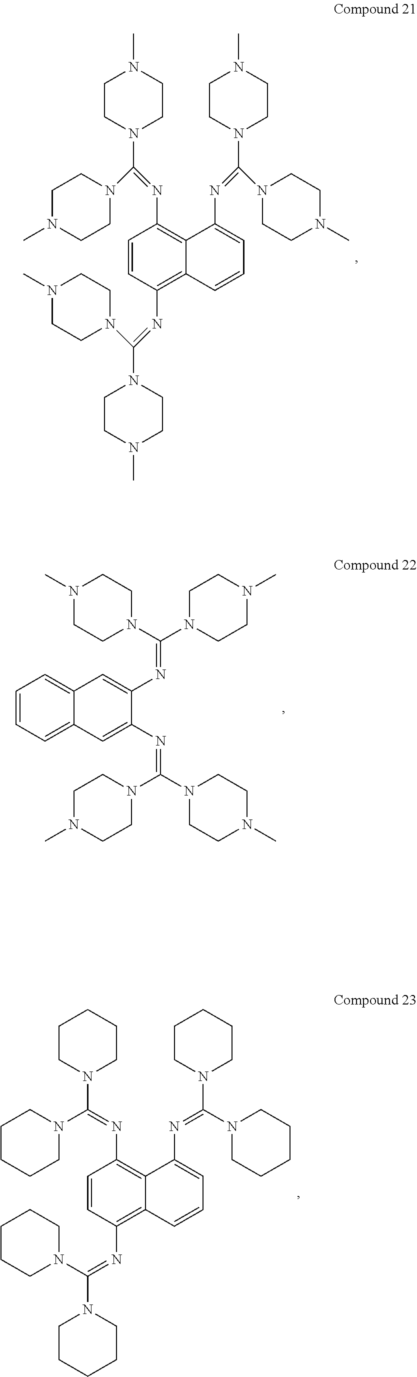

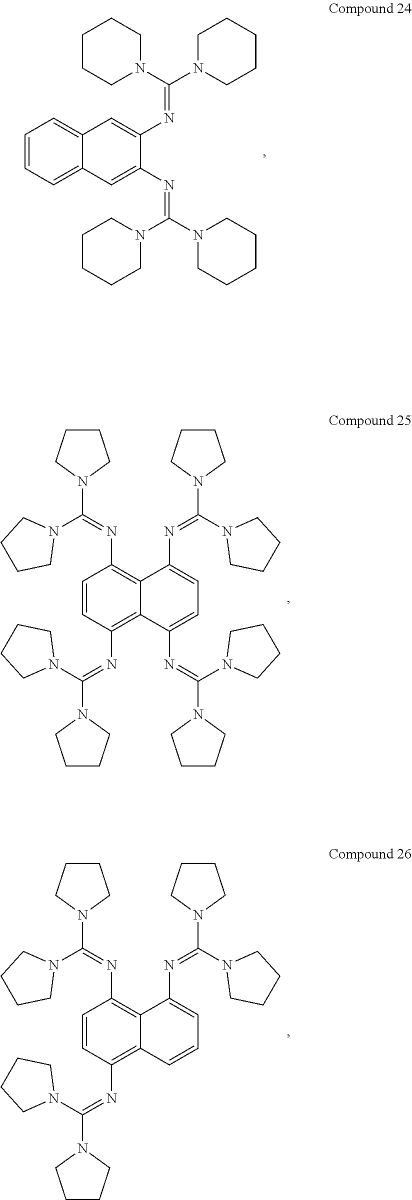 [SMILES: c1ccc2cc(N=C(N3CCCCC3)N3CCCCC3)c(N=C(N3CCCCC3)N3CCCCC3)cc2c1 ; c1cc(N=C(N2CCCC2)N2CCCC2)c2c(N=C(N3CCCC3)N3CCCC3)ccc(N=C(N3CCCC3)N3CCCC3)c2c1N=C(N1CCCC1)N1CCCC1 ; c1cc(N=C(N2CCCC2)N2CCCC2)c2c(N=C(N3CCCC3)N3CCCC3)ccc(N=C(N3CCCC3)N3CCCC3)c2c1]

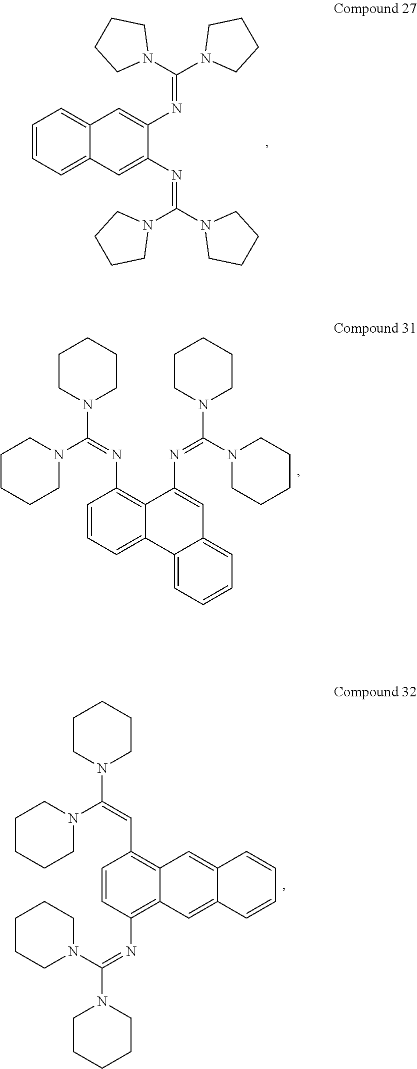 [SMILES: c1ccc2cc(N=C(N3CCCC3)N3CCCC3)c(N=C(N3CCCC3)N3CCCC3)cc2c1 ; c1ccc2c(c1)cc(N=C(N1CCCCC1)N1CCCCC1)c1c(N=C(N3CCCCC3)N3CCCCC3)cccc12 ; C(=C(N1CCCCC1)N1CCCCC1)c1ccc(N=C(N2CCCCC2)N2CCCCC2)c2cc3ccccc3cc12]

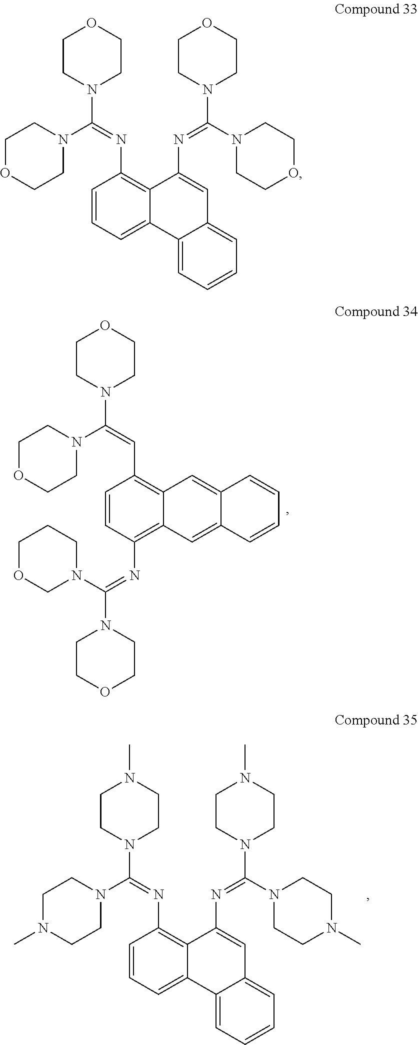

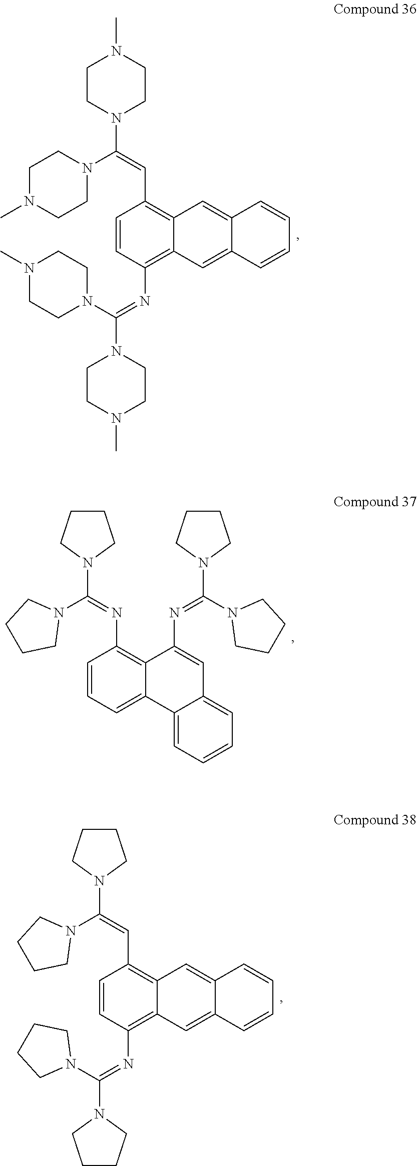 [SMILES: CN1CCN(C(=Cc2ccc(N=C(N3CCN(C)CC3)N3CCN(C)CC3)c3cc4ccccc4cc23)N2CCN(C)CC2)CC1 ; c1ccc2c(c1)cc(N=C(N1CCCC1)N1CCCC1)c1c(N=C(N3CCCC3)N3CCCC3)cccc12 ; C(=C(N1CCCC1)N1CCCC1)c1ccc(N=C(N2CCCC2)N2CCCC2)c2cc3ccccc3cc12]

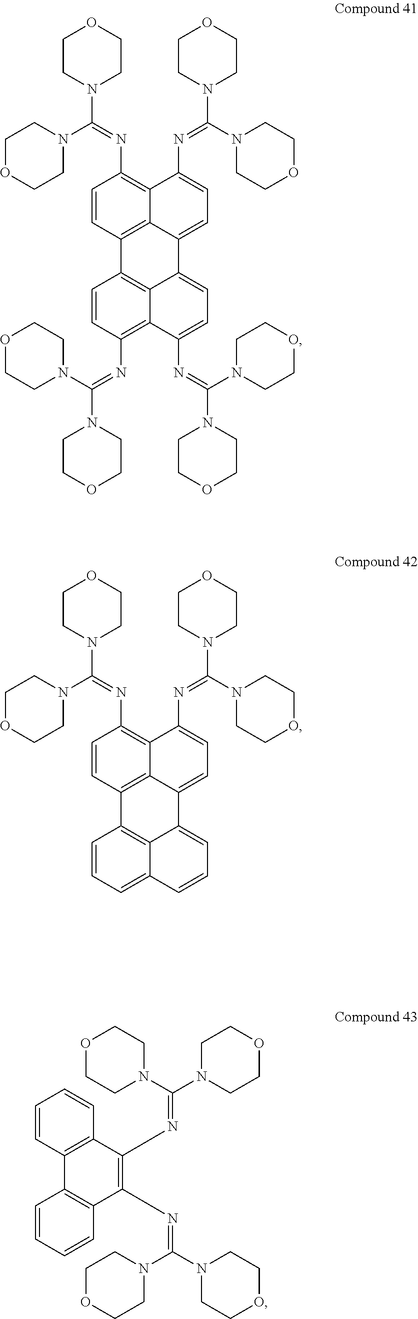 [SMILES: c1cc2c3ccc(N=C(N4CCOCC4)N4CCOCC4)c4c(N=C(N5CCOCC5)N5CCOCC5)ccc(c5ccc(N=C(N6CCOCC6)N6CCOCC6)c(c1N=C(N1CCOCC1)N1CCOCC1)c25)c43 ; c1cc2cccc3c4ccc(N=C(N5CCOCC5)N5CCOCC5)c5c(N=C(N6CCOCC6)N6CCOCC6)ccc(c(c1)c23)c54 ; c1ccc2c(c1)c(N=C(N1CCOCC1)N1CCOCC1)c(N=C(N1CCOCC1)N1CCOCC1)c1ccccc12]

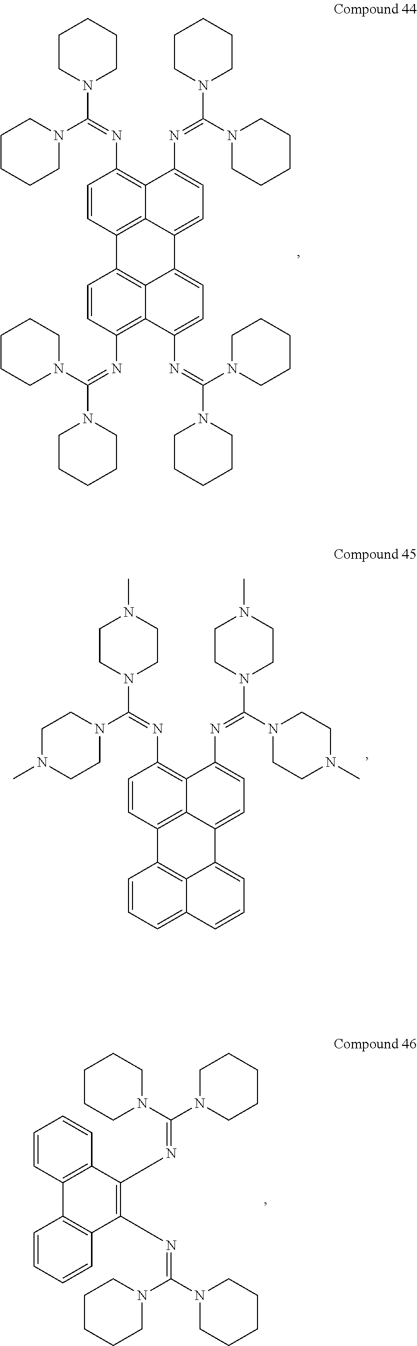

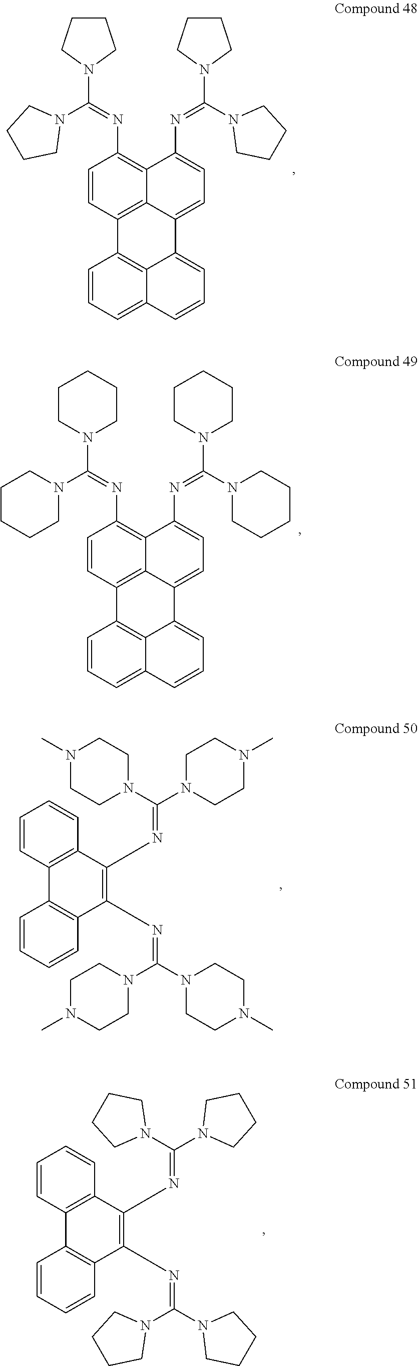 [SMILES: c1cc2cccc3c4ccc(N=C(N5CCCC5)N5CCCC5)c5c(N=C(N6CCCC6)N6CCCC6)ccc(c(c1)c23)c54 ; c1cc2cccc3c4ccc(N=C(N5CCCCC5)N5CCCCC5)c5c(N=C(N6CCCCC6)N6CCCCC6)ccc(c(c1)c23)c54 ; CN1CCN(C(=Nc2c(N=C(N3CCN(C)CC3)N3CCN(C)CC3)c3ccccc3c3ccccc23)N2CCN(C)CC2)CC1 ; c1ccc2c(c1)c(N=C(N1CCCC1)N1CCCC1)c(N=C(N1CCCC1)N1CCCC1)c1ccccc12]

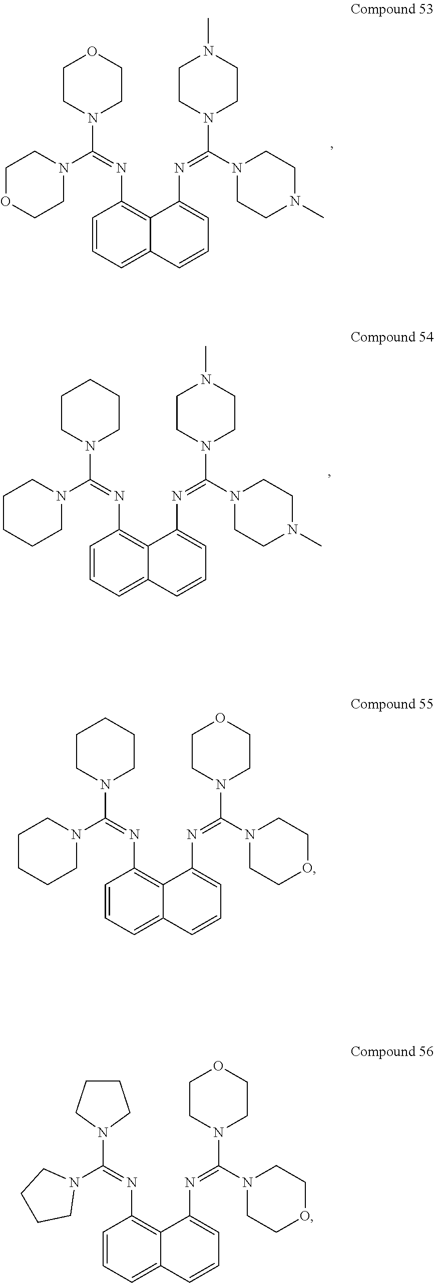 [SMILES: CN1CCN(C(=Nc2cccc3cccc(N=C(N4CCOCC4)N4CCOCC4)c23)N2CCN(C)CC2)CC1 ; CN1CCN(C(=Nc2cccc3cccc(N=C(N4CCCCC4)N4CCCCC4)c23)N2CCN(C)CC2)CC1 ; c1cc(N=C(N2CCCCC2)N2CCCCC2)c2c(N=C(N3CCOCC3)N3CCOCC3)cccc2c1 ; c1cc(N=C(N2CCCC2)N2CCCC2)c2c(N=C(N3CCOCC3)N3CCOCC3)cccc2c1]

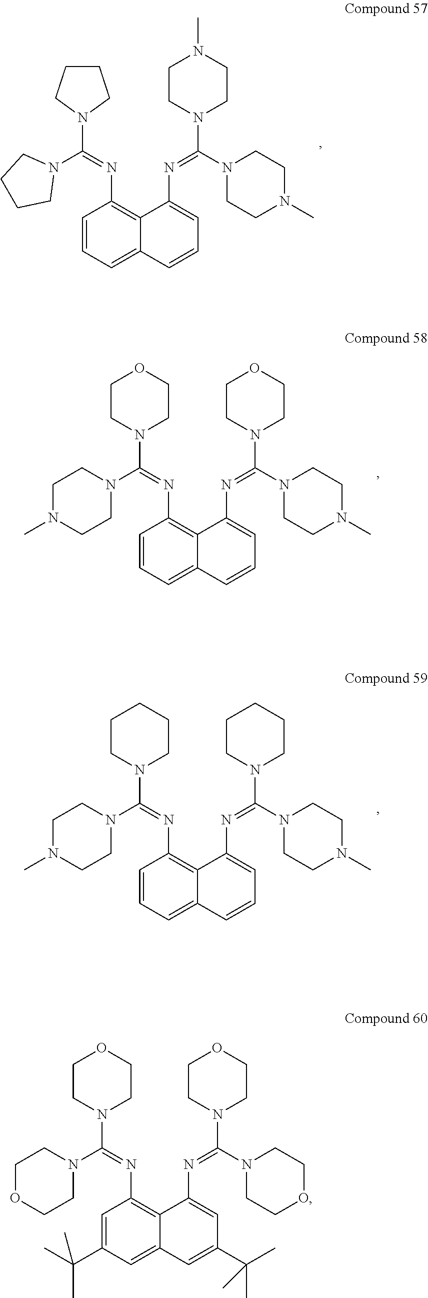

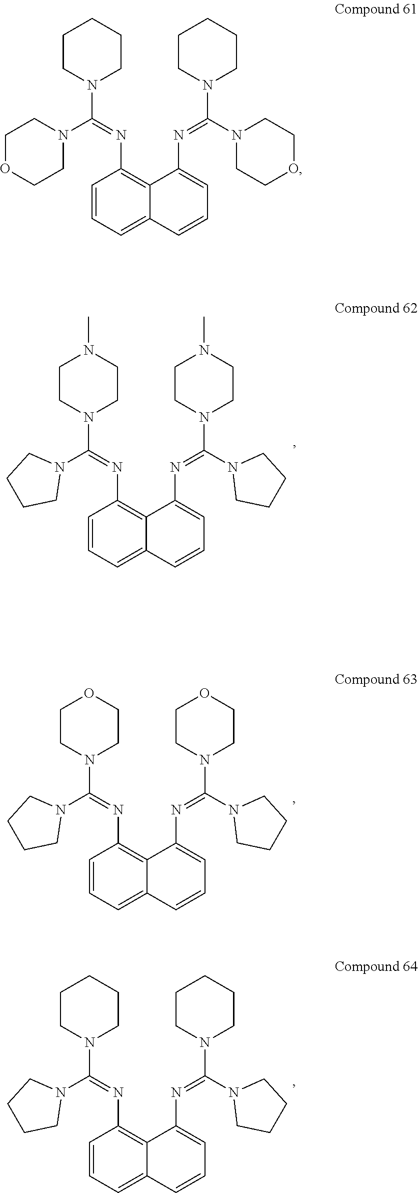 [SMILES: c1cc(/N=C(/N2CCCCC2)N2CCOCC2)c2c(/N=C(/N3CCCCC3)N3CCOCC3)cccc2c1 ; CN1CCN(/C(=N/c2cccc3cccc(/N=C(\N4CCCC4)N4CCN(C)CC4)c23)N2CCCC2)CC1 ; c1cc(/N=C(\N2CCCC2)N2CCOCC2)c2c(/N=C(\N3CCCC3)N3CCOCC3)cccc2c1 ; c1cc(/N=C(/N2CCCCC2)N2CCCC2)c2c(/N=C(/N3CCCCC3)N3CCCC3)cccc2c1]

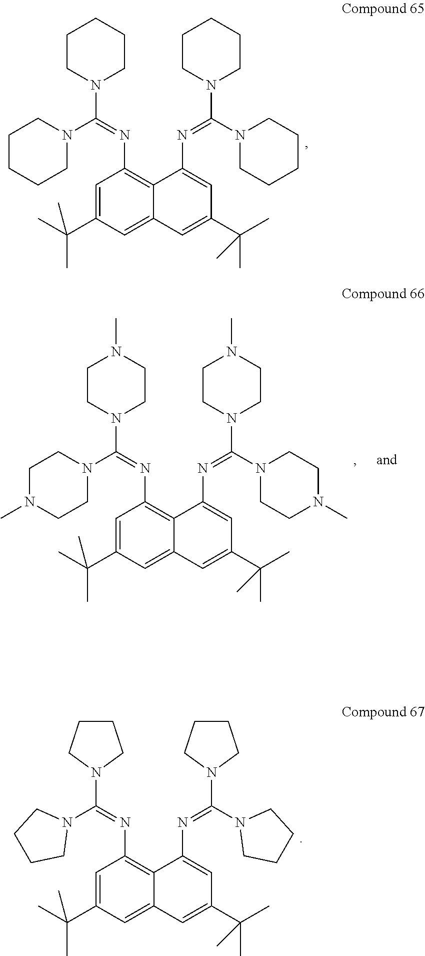

8. The use of the chemical compound as claimed in claim 1 in an electronic component, in particular in an organic electronic component, preferably as a dopant for doping layers in an organic electronic component, in particular at least one transport layer and/or injection layer.

9. An electronic component, preferably an organic electronic component, having an electrode, a counter-electrode and a layer system between the electrode and the counter-electrode, wherein the layer system comprises at least one organic layer, preferably at least one photoactive layer, and at least one transport layer, wherein the at least one organic layer and/or the at least one transport layer comprises at least one chemical compound as claimed in claim 1.

10. The electronic component as claimed in claim 9, wherein the proportion of the at least one chemical compound in the at least one organic layer and/or the at least one transport layer is in each case at most 35% by weight, preferably at most 30% by weight, or preferably at most 25% by weight, based on the total weight of the layer.
